(19) United States
(12) Patent Application Publication
(10) Pub. No.: US 2025/0201409 A1
(43) Pub. Date: Jun. 19, 2025

(54) DISEASE CLASSIFIERS FROM TARGETED MICROBIAL AMPLICON SEQUENCING

(71) Applicant: MICRONOMA, INC., San Diego, CA (US)

(72) Inventors: Serena FRARACCIO, San Diego, CA (US); Stephen WANDRO, San Diego, CA (US); Eddie ADAMS, San Diego, CA (US); Gregory D. POORE, San Diego, CA (US)

(21) Appl. No.: 18/845,660

(22) PCT Filed: Mar. 9, 2023
(86) PCT No.: PCT/US2023/064065

§ 371 Date: Sep. 10, 2024

Related U.S. Application Data
(60) Provisional application No. 63/318,479, filed on Mar. 10, 2022.

(51) Int. Cl. G16H 50/20; C12Q 1/686; C12Q 1/6869; C12Q 1/6886; C12Q 1/689; C12Q 1/6895; G06N 20/20; G16B 40/10; G16B 40/20; G16H 50/70

(52) U.S. Cl. CPC G16H 50/20; C12Q 1/686; C12Q 1/6869; C12Q 1/6886; C12Q 1/689; C12Q 1/6895; G06N 20/20; G16B 40/10; G16B 40/20; G16H 50/70

(57) ABSTRACT

Provided herein are multi-modal methods and systems of diagnosing one or more disease, as described elsewhere herein.

### CROSS REFERENCE

This application claims the benefit of U.S. Provisional Application No. 63/318,479 filed Mar. 10, 2022, which is hereby incorporated by reference for all purposes.

### SUMMARY OF THE INVENTION

Aspects of the disclosure provide a method of generating a feature set for differentiating cancer and non-cancer health states of one or more subjects. In some embodiments, the method is based on targeted amplicon sequencing of one or more microbial genomic features. In some embodiments, the method comprises the steps of: (a) providing one or more subjects' one or more nucleic acids and corresponding health states; (b) amplifying one or more genomic features of one or more non-mammalian nucleic acids of said one or more nucleic acids, thereby generating an amplified one or more genomic features; (c) sequencing said amplified one or more genomic features to generate one or more non-mammalian sequencing reads; and (d) generating a feature set configured to differentiate a cancer and non-cancer health state by combining said one or more genomic feature abundances of said one or more non-mammalian sequencing reads and said health state of said one or more subjects. In some embodiments, the genomic features comprise microbial phylogenetic marker genes or marker gene fragments thereof. In some embodiments, the microbial phylogenetic marker genes comprise bacterial marker genes or marker gene fragments thereof. In some embodiments, the microbial phylogenetic marker genes comprise fungal marker genes or marker gene fragments thereof. In some embodiments, the bacterial marker genes comprise: ribosomal RNA gene 5S, ribosomal RNA gene 16S, ribosomal RNA gene 23S, bacterial housekeeping genes dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, tsf, or any combination thereof. In some embodiments, the fungal marker genes comprise: ribosomal RNA gene 18S, ribosomal RNA gene 5.8S, ribosomal RNA gene 28S, the internal transcribed spacer regions 1 and 2, or any combination thereof. In some embodiments, the microbial phylogenetic marker genes comprise bacterial, fungal, or any combination thereof marker genes. In some embodiments, amplifying comprises performing a polymerase chain reaction or derivatives thereof. In some embodiments, polymerase chain reaction derivatives comprise inverse PCR, anchored PCR, primer-directed rolling circle amplification, or any combination thereof. In some embodiments, the polymerase chain reaction comprises blocking primers, marker gene primers, or any combination thereof, configured to prevent amplification of one or more genomic features. In some embodiments, the one or more genomic features comprise mitochondrial DNA genomic features. In some embodiments, the blocking primers inhibit amplification of mitochondrial DNA genomic features. In some embodiments, the method further comprises enriching the one or more nucleic acids. In some embodiments, the one or more nucleic acids comprise mammalian, non-mammalian, or any combination thereof nucleic acids. In some embodiments, nucleic acid enrichment comprises the steps of: (a) combining the one or more mammalian and non-mammalian nucleic acids with hybridization probes, where the hybridization probes comprise a nucleic acid sequence complementarity to non-mammalian genomic features; (b) incubating the hybridization probes and one or more mammalian and non-mammalian nucleic acids under conditions that promote nucleic acid base pairing between target nucleic acid features and said hybridization probes; (c) separating unbound hybridization probes and hybridized probes bound to non-mammalian nucleic acids; and (d) washing said hybridized probes bound to non-mammalian nucleic acids, thereby generating one or more enriched non-mammalian nucleic acids. In some embodiments, washing is configured to remove non-specifically associated nucleic acids and other reaction components. In some embodiments, the enrichment of the one or more nucleic acids comprises non-mammalian DNA enrichment. In some embodiments, non-mammalian DNA enrichment comprises the steps of: (a) combining the one or more mammalian and non-mammalian nucleic acids with one or more recombinant CXXC-domain proteins to form a protein-DNA binding reaction; (b) incubating the protein-DNA binding reaction under conditions that promote an interaction between the recombinant CXXC-domain proteins and non-methylated CpG motifs of the one or more mammalian or non-mammalian nucleic acids; (c) separating unbound recombinant CXXC-domain proteins and recombinant CXXC-domain proteins bound to the non-methylated CpG nucleic acid fragments from the remainder of the protein-DNA binding reaction; and (d) washing the recombinant CXXC-domain proteins bound to the non-methylated CpG nucleic acid fragments, thereby generating one or more enriched nucleic acids for amplification. In some embodiments, washing is configured to remove non-specifically associated nucleic acids and the remainder of protein-DNA binding reaction components. In some embodiments, the one or more nucleic acids are derived from one or more biological samples of said one or more subjects. In some embodiments, the one or more biological samples comprise a tissue, liquid, or any combination thereof biopsy sample. In some embodiments, the liquid biopsy sample comprises: plasma, serum, whole blood, urine, cerebral spinal fluid, saliva, sweat, tears, exhaled breath condensate, or any combination thereof. In some embodiments, the one or more subjects comprise human, non-human mammal, or any combination thereof subjects. In some embodiments, the mammalian and non-mammalian nucleic acids comprise: DNA, RNA, microbial cell free DNA, microbial cell free RNA, cell-free DNA, cell-free RNA, exosomal DNA, exosomal RNA, or any combination thereof nucleic acids. In some embodiments, the method comprises filtering the one or more non-mammalian sequencing reads. In some embodiments, filtering comprises filtering the one or more non-mammalian sequencing reads to produce one or more mitochondrial DNA-depleted non-mammalian sequencing reads. In some embodiments, filtering comprises mapping the one or more mitochondrial DNA-depleted non-mammalian sequencing reads against one or more microbial reference databases to determine microbial taxonomic identity of the one or more mitochondrial DNA-depleted non-mammalian sequencing reads. In some embodiments, the method comprises decontaminating the one or more mitochondrial DNA-depleted non-mammalian sequencing reads. In some embodiments, decontamination comprises in-silico decontamination. In some embodiments, decontamination is configured to remove non-endogenous microbial sequencing reads, thereby generating decontaminated microbial taxonomic assignments and associated quantity of sequencing reads. In some embodiments, non-mammalian sequencing read mapping is performed with QIIME2 or other supported versions thereof. In some embodiments, the one or more microbial reference databases comprise the bacterial 16S rRNA database Greengenes; the bacterial, fungal and archaeal rRNA database SILVA; the eukaryotic nuclear ribosomal ITS region database UNITE; a custom database derived from publicly available and complete microbial genome sequences; or any combination thereof. In some embodiments, the one or more genomic feature abundances of the one or more non-mammalian sequencing reads comprise microbial functional gene, biochemical pathway, or any combination thereof abundances. In some embodiments, the method comprises predicting metagenomic functional content of the decontaminated microbial taxonomic assignments, thereby producing one or more functional abundances. In some embodiments, predicting the metagenomic functional content is performed by PICRUSt2. In some embodiments, the cancer comprises lung, breast, ovarian, gastro-intestinal, head and neck, liver, pancreas, prostate, skin, or any combination thereof cancers. In some embodiments, lung cancer comprises non-small cell lung cancer. In some embodiments, the cancer comprises a cancer of stage I, II, or III. In some embodiments, the non-cancer state comprises healthy, disease, or any combination thereof non-cancer state. In some embodiments, the disease state comprises lung disease, wherein the lung disease comprises: carcinoid, hamartoma, granuloma, interstitial fibrosis, emphysema, bronchitis, chronic obstructive pulmonary disease, pneumonia, sarcoidosis, or any combination thereof. In some embodiments, the method comprises generating a trained predictive model is, where the trained predictive model is trained with the feature set and the health state of said one or more subjects. In some embodiments, the trained predictive model comprises a machine learning model, one or more machine learning models, an ensemble of machine learning models, or any combination thereof. In some embodiments, the trained predictive model comprises a regularized machine learning model. In some embodiments, machine learning model comprises a machine learning classifier. In some embodiments, the machine learning model comprises a gradient boosting machine, neural network, support vector machine, k-means, classification trees, random forest, regression, or any combination thereof machine learning models.

Aspects disclosed herein provide a method of using an output of a trained predictive model to diagnose a cancer or non-cancer health state of one or more subjects. In some embodiments, the method comprises the steps of: (a) providing one or more subjects' one or more nucleic acids; (b) amplifying one or more genomic features of one or more non-mammalian nucleic acids, thereby generating an amplified one or more genomic features; (c) sequencing the amplified one or more genomic features to generate one or more non-mammalian sequencing reads; and (d) outputting a diagnosis of a cancer or non-cancer health state of the one or more subjects at least as a result of providing the one or more genomic features as an input to a trained predictive model. In some embodiments, the non-mammalian nucleic acids comprise microbial nucleic acids. In some embodiments, the one or more nucleic acids are derived from one or more biological samples of the one or more subjects. In some embodiments, the one or more biological samples comprise: a tissue, liquid, or any combination thereof biopsy sample. In some cases, the liquid biopsy sample comprises: plasma, serum, whole blood, urine, cerebral spinal fluid, saliva, sweat, tears, exhaled breath condensate, or any combination thereof. In some embodiments, the one or more subjects comprise human, non-human mammal, or any combination thereof subjects. In some embodiments, the one or more nucleic acids comprise a total population of DNA, RNA, cell-free DNA, cell-free RNA, exosomal DNA, exosomal RNA, cell-free microbial DNA, cell-free microbial RNA, or any combination thereof. In some embodiments, the one or more genomic features comprise microbial phylogenetic marker genes or marker gene fragments thereof. In some embodiments, the microbial phylogenetic marker genes may comprise bacterial marker genes or marker gene fragments thereof. In some embodiments, the microbial phylogenetic marker genes comprise fungal marker genes or marker gene fragments thereof. In some embodiments, the bacterial marker genes comprise: ribosomal RNA gene 5S, ribosomal RNA gene 16S, ribosomal RNA gene 23S, bacterial housekeeping genes dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, tsf, or any combination thereof. In some embodiments, the fungal marker genes comprise: ribosomal RNA gene 18S, ribosomal RNA gene 5.8S, ribosomal RNA gene 28S, internal transcribed spacer regions 1 and 2, or any combination thereof. In some embodiments, the microbial phylogenetic marker genes comprise bacterial, fungal, or any combination thereof marker genes. In some embodiments, amplifying comprises performing a polymerase chain reaction or derivatives thereof. In some embodiments, polymerase chain reaction derivatives comprise: inverse PCR, anchored PCR, primer-directed rolling circle amplification, or any combination thereof. In some embodiments, the polymerase chain reaction comprises blocking primers, marker gene primers, or any combination thereof, configured to prevent amplification of one or more genomic features. In some embodiments, the one or more genomic features comprise mitochondrial DNA genomic features. In some embodiments, the blocking primers inhibit amplification of mitochondrial DNA genomic features. In some embodiments, the method comprises enriching the one or more nucleic acids. In some embodiments, the one or more nucleic acids comprise mammalian, non-mammalian, or any combination thereof nucleic acids. In some embodiments, the mammalian and non-mammalian nucleic acids comprise DNA, RNA, microbial cell free DNA, microbial cell free RNA, cell-free DNA, cell-free RNA, exosomal DNA, exosomal RNA, or any combination thereof nucleic acids. In some embodiments, nucleic acid enrichment comprises the steps of: (a) combining said one or more mammalian and non-mammalian nucleic acids with hybridization probes, where the hybridization probes comprise a nucleic acid sequence complementarity to non-mammalian genomic features; (b) incubating the hybridization probes and one or more mammalian and non-mammalian nucleic acids under conditions that promote nucleic acid base pairing between target nucleic acid features and said hybridization probes; (c) separating unbound hybridization probes and hybridized probes bound to non-mammalian nucleic acids; and (d) washing the hybridized probes bound to non-mammalian nucleic acids, thereby generating one or more enriched non-mammalian nucleic acids. In some embodiments, washing is configured to remove non-specifically associated nucleic acids and other reaction components. In some embodiments, the enrichment of said one or more nucleic acids comprises non-mammalian DNA enrichment. In some embodiments, non-mammalian DNA enrichment comprises the steps of: (a) combining the one or more mammalian and non-mammalian nucleic acids with one or more recombinant CXXC-domain proteins to form a protein-DNA binding reaction; (b) incubating the protein-DNA binding reaction under conditions that promote an interaction between the recombinant CXXC-domain proteins and non-methylated CpG motifs of the one or more mammalian or non-mammalian nucleic acids; (c) separating unbound recombinant CXXC-domain proteins and recombinant CXXC-domain proteins bound to the non-methylated CpG nucleic acid fragments from the remainder of the protein-DNA binding reaction; and (d) washing the recombinant CXXC-domain proteins bound to the non-methylated CpG nucleic acid fragments, thereby generating one or more enriched nucleic acids for amplification. In some embodiments, washing is configured to remove non-specifically associated nucleic acids and said remainder of protein-DNA binding reaction components. In some embodiments, the recombinant CXXC-domain proteins comprise: recombinant zinc finger CXXC domain-containing proteins KDM2A, KDM2A, KDM2B, FBXL19, CFP1, DNMT1, MLL1, MLL2, MDB1, TET1, TET3, IDAX, CXXC5, CGBP, the recombinant CXXC domains derived therefrom, or any combination thereof. In some embodiments, the method comprises filtering the one or more non-mammalian sequencing. In some embodiments, filtering comprises filtering the one or more non-mammalian sequencing reads to produce one or more mitochondrial DNA-depleted non-mammalian sequencing reads. In some embodiments, filtering comprises mapping the one or more mitochondrial DNA-depleted non-mammalian sequencing reads against one or more microbial reference databases to determine microbial taxonomic identity of the one or more mitochondrial DNA-depleted non-mammalian sequencing reads. In some embodiments, the method comprises decontaminating the one or more mitochondrial DNA-depleted non-mammalian sequencing reads. In some embodiments, the decontamination comprises in-silico decontamination. In some embodiments, decontamination is configured to remove non-endogenous microbial sequencing reads, thereby generating decontaminated microbial taxonomic assignments and associated quantity of sequencing reads. In some embodiments, non-mammalian sequencing read mapping is performed with QIIME2 or other supported versions thereof. In some embodiments, the one or more microbial reference databases comprise: the bacterial 16S rRNA database Greengenes; the bacterial, fungal and archaeal rRNA database SILVA; the eukaryotic nuclear ribosomal ITS region database UNITE; a custom database derived from publicly available and complete microbial genome sequences; or any combination thereof. In some embodiments, the one or more genomic feature comprises an abundances of the one or more non-mammalian sequencing reads' microbial functional genes, biochemical pathways, or any combination thereof abundances. In some embodiments, the method comprises predicting the metagenomic functional content of the decontaminated microbial taxonomic assignments, thereby producing one or more functional abundances. In some embodiments, metagenomic functional content is performed by PICRUSt2. In some embodiments, the cancer health state comprises: lung, breast, ovarian, gastro-intestinal, head and neck, liver, pancreas, prostate, skin, or any combination thereof cancers. In some embodiments, lung cancer comprises non-small cell lung cancer. In some embodiments, the cancer comprises a cancer of stage I, II, or III. In some embodiments, the non-cancer state comprises healthy, disease, or any combination thereof non-cancer state. In some embodiments, the disease state comprises lung disease, where the lung disease comprises: carcinoid, hamartoma, granuloma, interstitial fibrosis, emphysema, bronchitis, chronic obstructive pulmonary disease, pneumonia, sarcoidosis, or any combination thereof. In some embodiments, the trained predictive model is trained with a feature set and a health state of one or more subjects. In some embodiments, the trained predictive model comprises a machine learning model, one or more machine learning models, an ensemble of machine learning models, or any combination thereof. In some embodiments, the trained predictive model comprises a regularized machine learning model. In some embodiments, machine learning model comprises a machine learning classifier. In some embodiments, the machine learning model comprises a gradient boosting machine, neural network, support vector machine, k-means, classification trees, random forest, regression, or any combination thereof machine learning models.

Aspects disclosed herein provide a system for diagnosing a cancerous or non-cancerous health state of one or more subjects. In some embodiments, the system comprising: (a) a processor; and (b) a non-transitory computer readable storage medium including software configured to cause the processor to: (i) receive one or more subjects' one or more nucleic acid sequencing reads of the one or more subjects' biological samples, where the one or more nucleic acid sequencing reads comprise an amplified one or more genomic features of one or more non-mammalian nucleic acids; and (ii) output a diagnosis of a cancerous or non-cancerous health state of the one or more subjects at least as a result of providing the one or more non-mammalian nucleic acid sequencing reads' one or more genomic features as an input to a trained predictive model. In some embodiments, the non-mammalian nucleic acids may comprise microbial nucleic acids. In some embodiments, the one or more biological samples comprise a tissue, liquid, or any combination thereof biopsy samples. In some embodiments, the one or more subjects may comprise human, non-human mammal, or any combination thereof subjects. In some embodiments, the liquid biopsy sample comprises: plasma, serum, whole blood, urine, cerebral spinal fluid, saliva, sweat, tears, exhaled breath condensate, or any combination thereof. In some embodiments, the one or more nucleic acids comprise: DNA, RNA, cell-free DNA, cell-free RNA, exosomal DNA, exosomal RNA, cell-free microbial DNA, cell-free microbial RNA, or any combination thereof. In some embodiments, the genomic features may comprise microbial phylogenetic marker genes or marker gene fragments thereof. In some embodiments, the microbial phylogenetic marker genes may comprise bacterial marker genes or marker gene fragments thereof. In some embodiments, the microbial phylogenetic marker genes may comprise fungal marker genes or marker gene fragments thereof. In some embodiments, the bacterial marker genes comprise ribosomal RNA genes. In some embodiments, the ribosomal RNA genes comprise 5S, 16S, 23S, or any combination thereof ribosomal RNA genes. In some embodiments, the bacterial marker genes comprise: ribosomal RNA gene 5S, ribosomal RNA gene 16S, ribosomal RNA gene 23S, bacterial housekeeping genes dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, tsf, or any combination thereof. In some embodiments, the fungal marker genes comprise: ribosomal RNA gene 18S, ribosomal RNA gene 5.8S, ribosomal RNA gene 28S, internal transcribed spacer regions 1 and 2, or any combination thereof. In some embodiments, the microbial phylogenetic marker genes may comprise bacterial, fungal, or any combination thereof marker genes. In some embodiments, the amplified one or more genomic features of the one or more non-mammalian nucleic acids are amplified by polymerase chain reaction or derivatives thereof. In some embodiments, polymerase chain reaction derivatives comprise inverse PCR, anchored PCR, primer-directed rolling circle amplification, or any combination thereof. In some embodiments, the polymerase chain reaction comprises blocking primers, marker gene primers, or any combination thereof, configured to prevent amplification of one or more genomic features. In some embodiments, the one or more genomic features comprise mitochondrial DNA genomic features. In some embodiments, the blocking primers inhibit amplification of mitochondrial DNA genomic features. In some embodiments, the one or more nucleic acid sequencing reads comprise sequencing reads of one or more enriched nucleic acids. In some embodiments, the one or more nucleic acids may comprise mammalian, non-mammalian, or any combination thereof nucleic acids. In some embodiments, the one or more enriched nucleic acids are generated by: (a) combining the one or more mammalian and non-mammalian nucleic acids with hybridization probes, wherein the hybridization probes comprise a nucleic acid sequence complementarity to non-mammalian genomic features; (b) incubating the hybridization probes and one or more mammalian and non-mammalian nucleic acids under conditions that promote nucleic acid base pairing between target nucleic acid features and the hybridization probes; (c) separating unbound hybridization probes and hybridized probes bound to non-mammalian nucleic acids; and (d) washing the hybridized probes bound to non-mammalian nucleic acids, thereby generating one or more enriched non-mammalian nucleic acids. In some embodiments, washing is configured to remove non-specifically associated nucleic acids and other reaction components. In some embodiments, the one or more enriched nucleic acids are generated by non-mammalian DNA enrichment. In some embodiments, the non-mammalian enrichment comprises: (a) combining the one or more mammalian and non-mammalian nucleic acids with one or more recombinant CXXC-domain proteins to form a protein-DNA binding reaction; (b) incubating the protein-DNA binding reaction under conditions that promote an interaction between the recombinant CXXC-domain proteins and non-methylated CpG motifs of the one or more mammalian or non-mammalian nucleic acids; (c) separating unbound recombinant CXXC-domain proteins and recombinant CXXC-domain proteins bound to the non-methylated CpG nucleic acid fragments from the remainder of the protein-DNA binding reaction; and (d) washing the recombinant CXXC-domain proteins bound to the non-methylated CpG nucleic acid fragments, thereby generating one or more enriched nucleic acids for amplification. In some embodiments, washing is configured to remove non-specifically associated nucleic acids and the remainder of protein-DNA binding reaction components. In some embodiments, the recombinant CXXC-domain proteins comprise: recombinant zinc finger CXXC domain-containing proteins KDM2A, KDM2A, KDM2B, FBXL19, CFP1, DNMT1, MLL1, MLL2, MDB1, TET1, TET3, IDAX, CXXC5, CGBP, the recombinant CXXC domains derived therefrom, or any combination thereof. In some embodiments, the software configures the processor to filter the one or more nucleic acid sequencing reads. In some embodiments, filtering comprises filtering the one or more sequencing reads to produce one or more mitochondrial DNA-depleted non-mammalian sequencing reads. In some embodiments, filtering comprises mapping the one or more mitochondrial DNA-depleted non-mammalian sequencing reads against one or more microbial reference databases to determine microbial taxonomic identity of the one or more mitochondrial DNA-depleted non-mammalian sequencing reads. In some embodiments, the software configures the processor to decontaminate the one or more mitochondrial DNA-depleted non-mammalian sequencing reads. In some embodiments, the decontamination comprises in-silico decontamination. In some embodiments, decontamination is configured to remove non-endogenous microbial sequencing reads, thereby generating decontaminated microbial taxonomic assignments and associated quantity of sequencing reads. In some embodiments, mapping is performed with QIIME2 or other supported versions thereof. In some embodiments, the one or more microbial reference databases comprise: the bacterial 16S rRNA database Greengenes; the bacterial, fungal, and archaeal rRNA database SILVA; the eukaryotic nuclear ribosomal ITS region database UNITE; a custom database derived from publicly available and complete microbial genome sequences; or any combination thereof. In some embodiments, the amplified one or more genomic feature comprises an abundances of one or more non-mammalian sequencing reads' microbial functional genes, biochemical pathways, or any combination thereof abundances. In some embodiments, predicting metagenomic functional content is performed on decontaminated microbial taxonomic assignments, thereby producing one or more functional abundances. In some embodiments, the software configures the processor to predict metagenomic functional content of the decontaminated microbial taxonomic assignments, thereby producing one or more functional abundances. In some embodiments, predicting the metagenomic functional content is performed by PICRUSt2. In some embodiments, the cancerous health state comprises: lung, breast, ovarian, gastro-intestinal, head and neck, liver, pancreas, prostate, skin, or any combination thereof cancers. In some embodiments, lung cancer comprises non-small cell lung cancer. In some embodiments, the cancerous state comprises a cancer of stage I, II, or III. In some embodiments, the non-cancerous health state comprises healthy, disease, or any combination thereof non-cancerous states. In some embodiments, the disease state may comprise lung disease, where lung disease comprises: carcinoid, hamartoma, granuloma, interstitial fibrosis, emphysema, bronchitis, chronic obstructive pulmonary disease, pneumonia, sarcoidosis, or any combination thereof. In some embodiments, the trained predictive model is trained with one or more genomic feature sets and said health states of the one or more subjects. In some embodiments, the trained predictive model comprises a machine learning model, one or more machine learning models, an ensemble of machine learning models, or any combination thereof. In some embodiments, the trained predictive model comprises a regularized machine learning model. In some embodiments, machine learning model comprises a machine learning classifier. In some embodiments, the machine learning model comprises a gradient boosting machine, neural network, support vector machine, k-means, classification trees, random forest, regression, or any combination thereof machine learning models. In some embodiments, the cancerous health state comprises one or more types of cancer, one or more subtypes of cancer, stage of cancer, cancer prognosis, or any combination thereof. In some embodiments, the cancerous or non-cancerous health state comprise a category, tissue specific location of cancer or disease, or any combination thereof. In some embodiments, the trained predictive model is used to predict cancer therapy response of the one or more subjects. In some embodiments, the trained predictive model is utilized to select an optimal therapy for the one or more subjects. In some embodiments, the trained predictive model is utilized to longitudinally model a course of one or more cancers of one or more subjects' response to a therapy and to then adjust a treatment regimen. In some embodiments, the cancerous health state may comprise: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof cancers. In some embodiments, the trained predictive model may be configured to remove contaminate non-mammalian features while selectively retaining other non-contaminate non-mammalian features.

Aspects disclosed herein provide a method of generating a feature set for differentiating a cancer type of one or more subjects, the method comprising: (a) providing one or more subjects' one or more nucleic acids and corresponding health states; (b) amplifying one or more genomic features of one or more non-mammalian nucleic acids of the one or more nucleic acids, thereby generating an amplified one or more genomic features; (c) sequencing the amplified one or more genomic features to generate one or more non-mammalian sequencing reads; and (d) generating a feature set configured to differentiate a cancer type by combining the one or more genomic feature abundances of the one or more non-mammalian sequencing reads and the health state of said one or more subjects. In some embodiments, the genomic features comprise microbial phylogenetic marker genes or marker gene fragments thereof. In some embodiments, the microbial phylogenetic marker genes comprise bacterial marker genes or marker gene fragments thereof. In some embodiments, the microbial phylogenetic marker genes comprise fungal marker genes or marker gene fragments thereof. In some embodiments, the bacterial marker genes comprise: ribosomal RNA gene 5S, ribosomal RNA gene 16S, ribosomal RNA gene 23S, bacterial housekeeping genes dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, tsf, or any combination thereof. In some embodiments, the fungal marker genes comprise: ribosomal RNA gene 18S, ribosomal RNA gene 5.8S, ribosomal RNA gene 28S, the internal transcribed spacer regions 1 and 2, or any combination thereof. In some embodiments, the microbial phylogenetic marker genes comprise bacterial, fungal, or any combination thereof marker genes. In some embodiments, amplifying comprises performing a polymerase chain reaction or derivatives thereof. In some embodiments, polymerase chain reaction derivatives comprise inverse PCR, anchored PCR, primer-directed rolling circle amplification, or any combination thereof. In some embodiments, the polymerase chain reaction comprises blocking primers, marker gene primers, or any combination thereof, configured to prevent amplification of one or more genomic features. In some embodiments, the one or more genomic features comprise mitochondrial DNA genomic features. In some embodiments, the blocking primers inhibit amplification of mitochondrial DNA genomic features. In some embodiments, the method further comprises enriching the one or more nucleic acids. In some embodiments, the one or more nucleic acids comprise mammalian, non-mammalian, or any combination thereof nucleic acids. In some embodiments, nucleic acid enrichment comprises the steps of: (a) combining the one or more mammalian and non-mammalian nucleic acids with hybridization probes, where the hybridization probes comprise a nucleic acid sequence complementarity to non-mammalian genomic features; (b) incubating the hybridization probes and one or more mammalian and non-mammalian nucleic acids under conditions that promote nucleic acid base pairing between target nucleic acid features and said hybridization probes; (c) separating unbound hybridization probes and hybridized probes bound to non-mammalian nucleic acids; and (d) washing said hybridized probes bound to non-mammalian nucleic acids, thereby generating one or more enriched non-mammalian nucleic acids. In some embodiments, washing is configured to remove non-specifically associated nucleic acids and other reaction components. In some embodiments, the enrichment of the one or more nucleic acids comprises non-mammalian DNA enrichment. In some embodiments, non-mammalian DNA enrichment comprises the steps of: (a) combining the one or more mammalian and non-mammalian nucleic acids with one or more recombinant CXXC-domain proteins to form a protein-DNA binding reaction; (b) incubating the protein-DNA binding reaction under conditions that promote an interaction between the recombinant CXXC-domain proteins and non-methylated CpG motifs of the one or more mammalian or non-mammalian nucleic acids; (c) separating unbound recombinant CXXC-domain proteins and recombinant CXXC-domain proteins bound to the non-methylated CpG nucleic acid fragments from the remainder of the protein-DNA binding reaction; and (d) washing the recombinant CXXC-domain proteins bound to the non-methylated CpG nucleic acid fragments, thereby generating one or more enriched nucleic acids for amplification. In some embodiments, washing is configured to remove non-specifically associated nucleic acids and the remainder of protein-DNA binding reaction components. In some embodiments, the one or more nucleic acids are derived from one or more biological samples of said one or more subjects. In some embodiments, the one or more biological samples comprise a tissue, liquid, or any combination thereof biopsy sample. In some embodiments, the liquid biopsy sample comprises: plasma, serum, whole blood, urine, cerebral spinal fluid, saliva, sweat, tears, exhaled breath condensate, or any combination thereof. In some embodiments, the one or more subjects comprise human, non-human mammal, or any combination thereof subjects. In some embodiments, the mammalian and non-mammalian nucleic acids comprise: DNA, RNA, microbial cell free DNA, microbial cell free RNA, cell-free DNA, cell-free RNA, exosomal DNA, exosomal RNA, or any combination thereof nucleic acids. In some embodiments, the method comprises filtering the one or more non-mammalian sequencing reads. In some embodiments, filtering comprises filtering the one or more non-mammalian sequencing reads to produce one or more mitochondrial DNA-depleted non-mammalian sequencing reads. In some embodiments, filtering comprises mapping the one or more mitochondrial DNA-depleted non-mammalian sequencing reads against one or more microbial reference databases to determine microbial taxonomic identity of the one or more mitochondrial DNA-depleted non-mammalian sequencing reads. In some embodiments, the method comprises decontaminating the one or more mitochondrial DNA-depleted non-mammalian sequencing reads. In some embodiments, decontamination comprises in-silico decontamination. In some embodiments, decontamination is configured to remove non-endogenous microbial sequencing reads, thereby generating decontaminated microbial taxonomic assignments and associated quantity of sequencing reads. In some embodiments, non-mammalian sequencing read mapping is performed with QIIME2 or other supported versions thereof. In some embodiments, the one or more microbial reference databases comprise the bacterial 16S rRNA database Greengenes; the bacterial, fungal, and archaeal rRNA database SILVA; the eukaryotic nuclear ribosomal ITS region database UNITE; a custom database derived from publicly available and complete microbial genome sequences; or any combination thereof. In some embodiments, the one or more genomic feature abundances of the one or more non-mammalian sequencing reads comprise microbial functional gene, biochemical pathway, or any combination thereof abundances. In some embodiments, the method comprises predicting metagenomic functional content of the decontaminated microbial taxonomic assignments, thereby producing one or more functional abundances. In some embodiments, predicting the metagenomic functional content is performed by PICRUSt2. In some embodiments, the cancer comprises lung, breast, ovarian, gastro-intestinal, head and neck, liver, pancreas, prostate, skin, or any combination thereof cancers. In some embodiments, lung cancer comprises non-small cell lung cancer. In some embodiments, the cancer comprises a cancer of stage I, II, or III. In some embodiments, the method comprises generating a trained predictive model is, where the trained predictive model is trained with the feature set and the health state of said one or more subjects. In some embodiments, the trained predictive model comprises a machine learning model, one or more machine learning models, an ensemble of machine learning models, or any combination thereof. In some embodiments, the trained predictive model comprises a regularized machine learning model. In some embodiments, machine learning model comprises a machine learning classifier. In some embodiments, the machine learning model comprises a gradient boosting machine, neural network, support vector machine, k-means, classification trees, random forest, regression, or any combination thereof machine learning models.

Aspects of the disclosure provided herein describe a method of determining a disease of a subject, comprising: receiving a biological sample, electronic medical record information, and one or more radiologic images of a subject; sequencing one or more nucleic acid molecules isolated from the biological sample thereby generating one or more nucleic acid molecule sequencing reads; and determining a disease of the subject as an output of a predictive model when the predictive model is provided the subject's one or more nucleic acid molecule sequencing reads, electronic medical record information, and data derived from one or more radiologic images as an input. In some embodiments, the method further comprises identifying one or more protein biomarkers from the biological sample of the subject. In some embodiments, the predictive model is provided the one or more protein biomarkers from the biological sample of the subject. In some embodiments, the one or more protein biomarkers comprise carcinoembryonic antigen, osteopontin, cancer antigen 15-3, cancer antigen 19-9, cancer antigen 125, interleukin-8, prolactin, cytokeratin 19 fragment (CYFRA 21-1), MMP-9, STNFRII, MMP-7, Resistin, MPO, MCP-1, GRO, sVEGFR2, SKDR, sFlk-1, VEGF-A, VEGF-C, VEGF-D, HGF, CRp, MIF, PDGF, AB/bb, RANTES, SAA, TNFRII, or a combination thereof. In some embodiments, the disease comprises cancer or non-cancerous diseased. In some embodiments, the biological sample comprises a liquid biopsy, a tissue biopsy, or a combination thereof. In some embodiments, the one or more radiologic images comprise x-ray, computed tomography (CT), low dose computed tomography, magnetic resonance imaging (MRI), ultrasound, positron emission tomography, fluoroscopy, angiography, or any combination thereof images. In some embodiments, the cancer comprises a tumor mass with a diameter less than 3 centimeters. In some embodiments, sequencing comprises amplicon-based 16S rRNA sequencing. In some embodiments, the amplicon-based 16S rRNA sequencing sequences the V6 region of the one or more nucleic acid molecules. In some embodiments, the one or more nucleic acid molecules comprise mammalian RNA, mammalian DNA, mammalian cell-free DNA, mammalian cell-free RNA, mammalian exosomal DNA, mammalian exosomal RNA, non-human RNA, non-human DNA, non-human cell-free DNA, non-human cell-free RNA, non-human exosomal DNA, non-human exosomal RNA, circulating tumor DNA, circulating tumor RNA, or any combination thereof. In some embodiments, the liquid biopsy comprises plasma, serum, whole blood, urine, cerebral spinal fluid, saliva, sweat, tears, exhaled breath condensate, or any combination thereof. In some embodiments, the cancer comprises lung adenocarcinoma (LUAD, lung squamous cell carcinoma (LUSC), small cell lung cancer (SCLC), or any combination thereof. In some embodiments, the cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. In some embodiments, the method further comprises calculating one or more features of the one or more radiologic images, wherein the one or more features of the one or more radiologic images are provided as an input to the predictive model. In some embodiments, the one or more features comprise Brock cancer probability score, lesion diameter, lesion spiculation, lesion solidity, or any combination thereof. In some embodiments, the method further comprises mapping or aligning the one or more nucleic acid sequencing reads to a genome database to determine one or more human, non-human, or a combination thereof features of the one or more nucleic acid sequencing reads. In some embodiments, the genome database comprises a human genome database. In some embodiments, the predictive model comprises a machine learning model. In some embodiments, the predictive model comprises a neural network, convolutional neural network, logistic regression, random forest, supper vector machines, or any combination thereof. In some embodiments, the machine learning model comprises a machine learning classifier. In some embodiments, the machine learning model comprises a stacked machine learning model, one or more machine learning models, an ensemble machine learning model, or a combination thereof. In some embodiments, the predictive model is trained with leave one out verification. In some embodiments, the predictive model is configured to determine a stage of the cancer, anatomical origin of the cancer, or a combination thereof. In some embodiments, the stage of the cancer is stage I, stage II, stage III, or stage IV. In some embodiments, the method further comprises decontaminating the one or more nucleic acid molecule sequencing reads to produce one or more decontaminated nucleic acid molecule sequencing reads. In some embodiments, decontaminating comprises in silico decontamination, experimental control decontamination, or a combination thereof. In some embodiments, the predictive model determines the disease with an accuracy of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, sequencing comprises shotgun metagenomic sequencing, next generation sequencing, long read sequencing, or any combination thereof. In some embodiments, the method further comprises determining one or more features of the one or more nucleic acid molecule sequencing reads. In some embodiments, the one or more features of the one or more nucleic acid molecules comprises non-microbial taxonomic abundance, mammalian genomic coordinates, annotated genomic loci, mammalian functional gene and/or biochemical pathway abundances, or any combination thereof features, and a number of sequencing reads associated with said one or more features. In some embodiments, the predictive model is configured to differentiate cancer and a non-cancerous disease of the subject. In some embodiments, the mapping or aligning is completed with Deblur, Bowtie2, Kraken, or any combination thereof.

Another aspect of the disclosure provided herein describes a method, comprising: receiving a biological sample, electronic medical record information, data derived from one or more radiologic images, and a corresponding disease of one or more subjects; sequencing one or more nucleic acid molecules isolated from the biological sample thereby generating one or more nucleic acid molecule sequencing reads; and identifying one or more features of the one or more nucleic acid molecule sequencing reads, electronic medical record information, and the data derived from the one or more radiologic images that correspond to the disease of the one or more subjects. In some embodiments, identifying comprises aligning the one or more sequencing reads to a genome database. In some embodiments, the method further comprises training a predictive model with the one or more features of the nucleic acid molecule sequencing reads, electronic medical record information, and the data derived from the one or more radiologic images and the corresponding disease of the one or more subjects. In some embodiments, the disease comprises cancer or non-cancerous disease. In some embodiments, the method further comprises identifying one or more features of one or more protein biomarkers of the biological sample of the subject. In some embodiments, the one or more protein biomarkers comprise carcinoembryonic antigen, osteopontin, cancer antigen 15-3, cancer antigen 19-9, cancer antigen 125, interleukin-8, prolactin, cytokeratin 19 fragment (CYFRA 21-1)), MMP-9, sTNFRII, MMP-7, Resistin, MPO, MCP-1, GRO, sVEGFR2, sKDR, sFlk-1, VEGF-A, VEGF-C, VEGF-D, HGF, CRp, MIF, PDGF, AB/bb, RANTES, SAA, TNFRII, or a combination thereof. In some embodiments, the biological sample comprises a liquid biopsy, a tissue biopsy, or a combination thereof. In some embodiments, the one or more radiologic images comprise x-ray, computed tomography (CT), low dose computed tomography, magnetic resonance imaging (MRI), ultrasound, positron emission tomography, fluoroscopy, angiography, or any combination thereof images. In some embodiments, the cancer comprises a tumor mass with a diameter less than 3 centimeters. In some embodiments, sequencing comprises amplicon-based 16S rRNA sequencing. In some embodiments, the amplicon-based 16S rRNA sequencing sequences the V6 region of the one or more nucleic acid molecules. In some embodiments, the one or more nucleic acid molecules comprise mammalian RNA, mammalian DNA, mammalian cell-free DNA, mammalian cell-free RNA, mammalian exosomal DNA, mammalian exosomal RNA, non-human RNA, non-human DNA, non-human cell-free DNA, non-human cell-free RNA, non-human exosomal DNA, non-human exosomal RNA, circulating tumor DNA, circulating tumor RNA, or any combination thereof. In some embodiments, the liquid biopsy comprises plasma, serum, whole blood, urine, cerebral spinal fluid, saliva, sweat, tears, exhaled breath condensate, or any combination thereof. In some embodiments, the cancer comprises lung adenocarcinoma (LUAD, lung squamous cell carcinoma (LUSC), small cell lung cancer (SCLC), or any combination thereof. In some embodiments, the cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. In some embodiments, the one or more radiologic image features comprise Brock cancer probability score, lesion diameter, lesion spiculation, lesion solidity, or any combination thereof. In some embodiments, the method further comprises mapping or aligning the one or more nucleic acid sequencing reads to a genome database to determine one or more human, non-human, or a combination thereof features of the one or more nucleic acid sequencing reads. In some embodiments, the genome database comprises a human genome database. In some embodiments, the predictive model comprises a machine learning model. In some embodiments, the predictive model comprises a neural network, convolutional neural network, logistic regression, random forest, supper vector machines, or any combination thereof. In some embodiments, the machine learning model comprises a machine learning classifier. In some embodiments, the machine learning model comprises a stacked machine learning model, one or more machine learning models, an ensemble machine learning model, or a combination thereof. In some embodiments, the predictive model is trained with leave one out verification. In some embodiments, the predictive model is configured to determine a stage of the cancer, anatomical origin of the cancer, or a combination thereof. In some embodiments, the stage of the cancer is stage I, stage II, or stage III, or stage IV. In some embodiments, the method further comprises decontaminating the one or more nucleic acid molecule sequencing reads to produce one or more decontaminated nucleic acid molecule sequencing reads. In some embodiments, decontaminating comprises in silico decontamination, experimental control decontamination, or a combination thereof. In some embodiments, the predictive model determines the disease with an accuracy of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, sequencing comprises shotgun sequencing, next generation sequencing, long read sequencing, or any combination thereof. In some embodiments, the method further comprising determining one or more features of the one or more nucleic acid molecule sequencing reads. In some embodiments, the one or more features of the one or more nucleic acid molecules comprises non-microbial taxonomic abundance, mammalian genomic coordinates, annotated genomic loci, mammalian functional gene and/or biochemical pathway abundances, or any combination thereof features, and a number of sequencing reads associated with said one or more features. In some embodiments, the predictive model is configured to differentiate cancer and a non-cancerous disease of the subject. In some embodiments, the mapping or aligning is completed with Deblur, Bowtie2, Kraken, or any combination thereof.

Another aspect of the disclosure provided herein describes a computer system configured to determine a disease of a subject, comprising: (a) one or more processors; and (b) a non-transient computer readable storage medium including software, wherein the software comprises executable instructions that, as a result of execution, cause the one or more processors of the computer system to: (i) receive one or more sequencing reads of a biological sample, electronic medical record information, and one or more images of a subject; and (ii) determine a disease of the subject as an output of a predictive model when the predictive model is provided the subject's one or more nucleic acid molecule sequencing reads, electronic medical record information, and data derived from one or more radiologic images as an input. In some embodiments, the disease comprises cancer or non-cancerous disease. In some embodiments, the biological sample comprises a tissue biopsy, liquid biopsy, or a combination thereof. In some embodiments, the executable instructions comprise receiving one or more protein biomarkers from the biological sample of the subject. In some embodiments, the predictive model is provided the one or more protein biomarkers from the biological sample of the subject. In some embodiments, the one or more protein biomarkers comprise carcinoembryonic antigen, osteopontin, or a combination thereof. In some embodiments, the predictive model is trained with the one or more features of the nucleic acid molecule sequencing reads, electronic medical record information, and the data derived from the one or more radiologic images and the corresponding disease of the one or more subjects. In some embodiments, the executable instructions comprise identifying one or more features of one or more protein biomarkers of the biological sample of the subject. In some embodiments, the one or more protein biomarkers comprise carcinoembryonic antigen, osteopontin, cancer antigen 15-3, cancer antigen 19-9, cancer antigen 125, interleukin-8, prolactin, cytokeratin 19 fragment (CYFRA 21-1), MMP-9, STNFRII, MMP-7, Resistin, MPO, MCP-1, GRO, SVEGFR2, SKDR, sFlk-1, VEGF-A, VEGF-C, VEGF-D, HGF, CRp, MIF, PDGF, AB/bb, RANTES, SAA, TNFRII, or a combination thereof. In some embodiments, the one or more radiologic images comprise x-ray, computed tomography (CT), low dose computed tomography, magnetic resonance imaging (MRI), ultrasound, positron emission tomography, fluoroscopy, angiography, or any combination thereof images. In some embodiments, the cancer comprises a tumor mass with a diameter less than 3 centimeters. In some embodiments, the one or more nucleic acid molecule sequencing reads comprises one or more amplicon-based 16S rRNA sequencing reads. In some embodiments, the amplicon-based 16S rRNA sequencing reads comprise sequencing reads of the V6 region of the one or more nucleic acid molecules. In some embodiments, the one or more nucleic acid molecule sequencing reads comprise sequencing reads of mammalian RNA, mammalian DNA, mammalian cell-free DNA, mammalian cell-free RNA, mammalian exosomal DNA, mammalian exosomal RNA, non-human RNA, non-human DNA, non-human cell-free DNA, non-human cell-free RNA, non-human exosomal DNA, non-human exosomal RNA, circulating tumor DNA, circulating tumor RNA, or any combination thereof. In some embodiments, the liquid biopsy comprises plasma, serum, whole blood, urine, cerebral spinal fluid, saliva, sweat, tears, exhaled breath condensate, or any combination thereof. In some embodiments, the cancer comprises lung adenocarcinoma (LUAD, lung squamous cell carcinoma (LUSC), small cell lung cancer (SCLC), or any combination thereof. In some embodiments, the cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. In some embodiments, the one or more radiologic image features comprise Brock cancer probability score, lesion diameter, lesion spiculation, lesion solidity, or any combination thereof. In some embodiments, the executable instructions further comprising mapping or aligning the one or more nucleic acid sequencing reads to a genome database to determine one or more human, non-human, or a combination thereof features of the one or more nucleic acid sequencing reads. In some embodiments, the genome database comprises a human genome database. In some embodiments, the predictive model comprises a machine learning model. In some embodiments, the predictive model comprises a neural network, convolutional neural network, logistic regression, random forest, supper vector machines, or any combination thereof. In some embodiments, the machine learning model comprises a machine learning classifier. In some embodiments, the machine learning model comprises a stacked machine learning model, one or more machine learning models, an ensemble machine learning model, or a combination thereof. In some embodiments, the predictive model is trained with leave one out verification. In some embodiments, the predictive model is configured to determine a stage of the cancer, anatomical origin of the cancer, or a combination thereof. In some embodiments, the stage of the cancer is stage I, stage II, stage III, or stage IV. In some embodiments, the executable instructions further comprise decontaminating the one or more nucleic acid molecule sequencing reads to produce one or more decontaminated nucleic acid molecule sequencing reads. In some embodiments, decontaminating comprises in silico decontamination, experimental control decontamination, or a combination thereof. In some embodiments, the predictive model determines the disease with an accuracy of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the one or more sequencing reads are generated by shotgun sequencing, next generation sequencing, long read sequencing, or any combination thereof. In some embodiments, the executable instructions further comprise determining one or more features of the one or more nucleic acid molecule sequencing reads. In some embodiments, the one or more features of the one or more nucleic acid molecules comprises non-microbial taxonomic abundance, mammalian genomic coordinates, annotated genomic loci, mammalian functional gene and/or biochemical pathway abundances, or any combination thereof features, and a number of sequencing reads associated with said one or more features. In some embodiments, the predictive model is configured to differentiate cancer and a non-cancerous disease of the subject. In some embodiments, the mapping or aligning is completed with Deblur, Bowtie2, Kraken, or any combination thereof.

Another aspect of the disclosure provided herein describes a method of determining a disease of a subject, comprising: receiving a biological sample from a subject; sequencing one or more nucleic acid molecules of the biological sample thereby generating one or more nucleic acid molecule sequencing reads; and determining a disease of the subject as an output of a predictive model when the predictive model is provided the subject's one or more nucleic acid molecule sequencing reads, wherein the predictive model is trained with one or more nucleic acid molecule sequencing reads of one or more liquid biological samples and one or more tissue biological samples and corresponding disease of one or more subjects. In some embodiments, the disease comprises cancer, non-cancerous diseased, or a combination thereof. In some embodiments, the method further comprises identifying one or more protein biomarkers from the biological sample of the subject. In some embodiments, the predictive model is provided the one or more protein biomarkers from the biological sample of the subject. In some embodiments, the one or more protein biomarkers comprise carcinoembryonic antigen, osteopontin, cancer antigen 15-3, cancer antigen 19-9, cancer antigen 125, interleukin-8, prolactin, cytokeratin 19 fragment (CYFRA 21-1), MMP-9, sTNFRII, MMP-7, Resistin, MPO, MCP-1, GRO, sVEGFR2, sKDR, sFlk-1, VEGF-A, VEGF-C, VEGF-D, HGF, CRp, MIF, PDGF, AB/bb, RANTES, SAA, TNFRII, or a combination thereof. In some embodiments, the cancer comprises a tumor mass with a diameter less than 3 centimeters millimeters. In some embodiments, the sequencing comprises amplicon-based 16S rRNA sequencing. In some embodiments, the amplicon-based 16S rRNA sequencing sequences the V6 region of the one or more nucleic acid molecules. In some embodiments, the one or more nucleic acid molecules comprise mammalian RNA, mammalian DNA, mammalian cell-free DNA, mammalian cell-free RNA, mammalian exosomal DNA, mammalian exosomal RNA, non-human RNA, non-human DNA, non-human cell-free DNA, non-human cell-free RNA, non-human exosomal DNA, non-human exosomal RNA, circulating tumor DNA, circulating tumor RNA, or any combination thereof. In some embodiments, the liquid biopsy comprises plasma, serum, whole blood, urine, cerebral spinal fluid, saliva, sweat, tears, exhaled breath condensate, or any combination thereof. In some embodiments, the cancer comprises lung adenocarcinoma (LUAD, lung squamous cell carcinoma (LUSC), small cell lung cancer (SCLC), or any combination thereof. In some embodiments, the cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. In some embodiments, the method further comprises mapping or aligning the one or more nucleic acid sequencing reads to a genome database to determine one or more human, non-human, or a combination thereof features of the one or more nucleic acid sequencing reads that are provided as an input to the predictive model. In some embodiments, the genome database comprises a human genome database. In some embodiments, the predictive model comprises a machine learning model. In some embodiments, the predictive model comprises a neural network, convolutional neural network, logistic regression, random forest, supper vector machines, or any combination thereof. In some embodiments, the machine learning model comprises a machine learning classifier. In some embodiments, the machine learning model comprises a stacked machine learning model, one or more machine learning models, an ensemble machine learning model, or a combination thereof. In some embodiments, the predictive model is trained with leave one out verification. In some embodiments, the predictive model is configured to determine a stage of the cancer, anatomical origin of the cancer, or a combination thereof. In some embodiments, the stage of the cancer is stage I, stage II, stage III, or stage IV. In some embodiments, the method further comprises decontaminating the one or more nucleic acid molecule sequencing reads to produce one or more decontaminated nucleic acid molecule sequencing reads, wherein the one or more decontaminated nucleic acid molecules are provided to the predictive model as an input. In some embodiments, decontaminating comprises in silico decontamination, experimental control decontamination, or a combination thereof. In some embodiments, the predictive model determines the disease with an accuracy of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, sequencing comprises shotgun sequencing, next generation sequencing, long read sequencing, or any combination thereof. In some embodiments, the method further comprises determining one or more features of the one or more nucleic acid molecule sequencing reads. In some embodiments, the one or more features of the one or more nucleic acid molecules comprises non-microbial taxonomic abundance, mammalian genomic coordinates, annotated genomic loci, mammalian functional gene and/or biochemical pathway abundances, or any combination thereof features, and a number of sequencing reads associated with said one or more features. In some embodiments, the predictive model is configured to differentiate cancer and a non-cancerous disease of the subject. In some embodiments, mapping or aligning is completed with Deblur, PICRUSt2, Bowtie2, Kraken, or any combination thereof.

Another aspect of the disclosure provided herein describes a method of identifying one or more non-human genomic features, comprising: receiving one or more liquid biological samples, one or more tissue biological samples, and a corresponding disease of one or more subjects; sequencing one or more nucleic acid molecules of the one or more liquid biological samples and the one or more tissue biological samples thereby generating one or more sequencing reads; and identifying one or more non-human genomic features that correspond to the disease of the one or more subjects from the one or more sequencing reads. In some embodiments, identifying comprises aligning or mapping the one or more sequencing reads to a genome database to determine one or more human, non-human, or a combination thereof features of the one or more nucleic acid sequencing reads. In some embodiments, the method further comprises training a predictive model with the one or more non-human genomic features and the corresponding disease of the one or more subjects. In some embodiments, the disease comprises cancer or non-cancerous disease. In some embodiments, the method further comprises identifying one or more features of one or more protein biomarkers of the one or more liquid biological sample, one or more tissue biological samples, or a combination thereof. In some embodiments, the one or more protein biomarkers comprise carcinoembryonic antigen, osteopontin, cancer antigen 15-3, cancer antigen 19-9, cancer antigen 125, interleukin-8, prolactin, cytokeratin 19 fragment (CYFRA 21-1), MMP-9, sTNFRII, MMP-7, Resistin, MPO, MCP-1, GRO, SVEGFR2, sKDR, sFlk-1, VEGF-A, VEGF-C, VEGF-D, HGF, CRp, MIF, PDGF, AB/bb, RANTES, SAA, TNFRII, or a combination thereof. In some embodiments, the cancer comprises a tumor mass with a diameter less than 3 centimeters. In some embodiments, the sequencing comprises amplicon-based 16S rRNA sequencing. In some embodiments, the amplicon-based 16S rRNA sequencing sequences the V6 region of the one or more nucleic acid molecules. In some embodiments, the one or more nucleic acid molecules comprise mammalian RNA, mammalian DNA, mammalian cell-free DNA, mammalian cell-free RNA, mammalian exosomal DNA, mammalian exosomal RNA, non-human RNA, non-human DNA, non-human cell-free DNA, non-human cell-free RNA, non-human exosomal DNA, non-human exosomal RNA, circulating tumor DNA, circulating tumor RNA, or any combination thereof. In some embodiments, the liquid biological sample comprises plasma, serum, whole blood, urine, cerebral spinal fluid, saliva, sweat, tears, exhaled breath condensate, or any combination thereof. In some embodiments, the cancer comprises lung adenocarcinoma (LUAD, lung squamous cell carcinoma (LUSC), small cell lung cancer (SCLC), or any combination thereof. In some embodiments, the cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. In some embodiments, the genome database comprises a human genome database. In some embodiments, the predictive model comprises a machine learning model. In some embodiments, the predictive model comprises a neural network, convolutional neural network, logistic regression, random forest, supper vector machines, or any combination thereof. In some embodiments, the machine learning model comprises a machine learning classifier. In some embodiments, the machine learning model comprises a stacked machine learning model, one or more machine learning models, an ensemble machine learning model, or a combination thereof. In some embodiments, the predictive model is trained with leave one out verification. In some embodiments, the predictive model is configured to determine a stage of the cancer, anatomical origin of the cancer, or a combination thereof. In some embodiments, the stage of the cancer is stage I, stage II, stage III, or stage IV. In some embodiments, the method further comprises decontaminating the one or more nucleic acid molecule sequencing reads to produce one or more decontaminated nucleic acid molecule sequencing reads. In some embodiments, decontaminating comprises in silico decontamination, experimental control decontamination, or a combination thereof. In some embodiments, the predictive model determines the disease with an accuracy of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, sequencing comprises shotgun sequencing, next generation sequencing, long read sequencing, or any combination thereof. In some embodiments, the method further comprises determining one or more features of the one or more nucleic acid molecule sequencing reads. In some embodiments, the one or more features of the one or more nucleic acid molecules comprises non-microbial taxonomic abundance, mammalian genomic coordinates, annotated genomic loci, mammalian functional gene and/or biochemical pathway abundances, or any combination thereof features, and a number of sequencing reads associated with said one or more features. In some embodiments, the predictive model is configured to differentiate cancer and a non-cancerous disease of the subject. In some embodiments, mapping or aligning is completed with Deblur, PICRUSt2, Bowtie2, Kraken, or any combination thereof.

Another aspect of the disclosure provided herein describes a computer system configured to determine a disease of a subject, comprising: (a) one or more processors; and (b) a non-transient computer readable storage medium including software, wherein the software comprises executable instructions that, as a result of execution, cause the one or more processors of the computer system to: (i) receive one or more sequencing reads of a biological samples of a subject; and (ii) determining a disease of the subject as an output of a predictive model when the predictive model is provided the subject's one or more nucleic acid molecule sequencing reads, wherein the predictive model is trained with one or more nucleic acid molecule sequencing reads of one or more liquid biological samples and one or more tissue biological samples and corresponding disease of one or more subjects. In some embodiments, the disease comprises cancer or non-cancerous disease. In some embodiments, the disease comprises cancer or non-cancerous disease. In some embodiments, the executable instructions comprise receiving one or more protein biomarkers from the biological sample of the subject. In some embodiments, the predictive model is provided the one or more protein biomarkers from the biological sample of the subject. In some embodiments, the executable instructions comprise identifying one or more features of one or more protein biomarkers of the biological sample of the subject. In some embodiments, the one or more protein biomarkers comprise carcinoembryonic antigen, osteopontin, cancer antigen 15-3, cancer antigen 19-9, cancer antigen 125, interleukin-8, prolactin, cytokeratin 19 fragment (CYFRA 21-1), MMP-9, sTNFRII, MMP-7, Resistin, MPO, MCP-1, GRO, sVEGFR2, sKDR, sFlk-1, VEGF-A, VEGF-C, VEGF-D, HGF, CRp, MIF, PDGF, AB/bb, RANTES, SAA, TNFRII, or a combination thereof. In some embodiments, the cancer comprises a tumor mass with a diameter less than 3 centimeters. In some embodiments, the one or more nucleic acid molecule sequencing reads comprises one or more amplicon-based 16S rRNA sequencing reads. In some embodiments the amplicon-based 16S rRNA sequencing reads comprise sequencing reads of the V6 region of the one or more nucleic acid molecules. In some embodiments, the one or more nucleic acid molecule sequencing reads comprise sequencing reads of mammalian RNA, mammalian DNA, mammalian cell-free DNA, mammalian cell-free RNA, mammalian exosomal DNA, mammalian exosomal RNA, non-human RNA, non-human DNA, non-human cell-free DNA, non-human cell-free RNA, non-human exosomal DNA, non-human exosomal RNA, circulating tumor DNA, circulating tumor RNA, or any combination thereof. In some embodiments, the liquid biological sample comprises plasma, serum, whole blood, urine, cerebral spinal fluid, saliva, sweat, tears, exhaled breath condensate, or any combination thereof. In some embodiments, the cancer comprises lung adenocarcinoma (LUAD, lung squamous cell carcinoma (LUSC), small cell lung cancer (SCLC), or any combination thereof. In some embodiments, the cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. In some embodiments, the executable instructions further comprising mapping or aligning the one or more nucleic acid sequencing reads to a genome database to determine one or more human, non-human, or a combination thereof features of the one or more nucleic acid sequencing reads. In some embodiments, the genome database comprises a human genome database. In some embodiments, the predictive model comprises a machine learning model. In some embodiments, the predictive model comprises a neural network, convolutional neural network, logistic regression, random forest, supper vector machines, or any combination thereof. In some embodiments, the machine learning model comprises a machine learning classifier. In some embodiments, the machine learning model comprises a stacked machine learning model, one or more machine learning models, an ensemble machine learning model, or a combination thereof. In some embodiments, the predictive model is trained with leave one out verification. In some embodiments, the predictive model is configured to determine a stage of the cancer, anatomical origin of the cancer, or a combination thereof. In some embodiments, the stage of the cancer is stage I, stage II, stage III, or stage IV. In some embodiments, the executable instructions further comprise decontaminating the one or more nucleic acid molecule sequencing reads to produce one or more decontaminated nucleic acid molecule sequencing reads. In some embodiments, decontaminating comprises in silico decontamination, experimental control decontamination, or a combination thereof. In some embodiments, the predictive model determines the disease with an accuracy of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the one or more sequencing reads are generated by shotgun sequencing, next generation sequencing, long read sequencing, or any combination thereof. In some embodiments, the executable instructions further comprise determining one or more features of the one or more nucleic acid molecule sequencing reads. In some embodiments, the one or more features of the one or more nucleic acid molecules comprises non-microbial taxonomic abundance, mammalian genomic coordinates, annotated genomic loci, mammalian functional gene and/or biochemical pathway abundances, or any combination thereof features, and a number of sequencing reads associated with said one or more features. In some embodiments, the predictive model is configured to differentiate cancer and a non-cancerous disease of the subject. In some embodiments, the mapping or aligning is completed with Deblur, PICRUSt2, Bowtie2, Kraken, or any combination thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a flow diagram of microbial nucleic acid amplification and/or enrichment method, as described in some embodiments herein.

FIG. 2 shows a flow diagram of a microbial taxonomy computational method, as described in some embodiments herein.

FIG. 3 shows a flow diagram of a microbial functional annotated computational method, as described in some embodiments herein.

FIG. 4 shows a flow diagram for a method of generating one or more microbial taxonomy based predictive model classifiers from nucleic acid samples of healthy, cancer, and/or non-cancerous non-healthy subjects.

FIG. 5 shows a flow diagram for a method of generating one or more microbial functional annotation predictive model classifiers from nucleic acid samples of healthy, cancer, and/or non-cancerous non-healthy subjects.

FIG. 6 shows a system configured to carry out the methods of the disclosure provided herein, as described in some embodiments herein.

FIGS. 7A-7B show 16S ribosomal RNA hypervariable regions and corresponding 16S primer used to amplify 16S regions of phylogenetically diverse bacteria, as described in some embodiments herein.

FIG. 8 shows a schematic representation of fungal ribosome RNA gene clusters with internally transcribed (ITS) regions, as described in some embodiments herein.

FIG. 9 shows experimental data of 16S ribosomal DNA amplification with a V6 primer pair and the microbial DNA standard composition amplified with said V6 primer pair, as described in some embodiments herein.

FIG. 10 shows experimental data of microbial 16S ribosomal DNA amplification with a V6 primer pair with and without the presence of human genomic DNA.

FIG. 11 shows experimental data of the specificity of 16S ribosomal DNA amplification with a V6 primer pair, as described in some embodiments herein.

FIGS. 12A-B shows a flow diagram for 16S sequencing library preparation (FIG. 12A) and western validation (FIG. 12B), as described in some embodiments herein.

FIG. 13 shows a flow diagram for 16S sequencing processing, as described in some embodiments herein.

FIGS. 14A-14B show experimental data sequencing read counts at various points through the 16S sequencing processing, as described in some embodiments herein.

FIGS. 15A-15B show a receiver operating characteristic curve for a trained predictive models in differentiating between non-small cell lung cancer and non-cancer nucleic acid samples from one or more subjects, as described elsewhere herein.

### DETAILED DESCRIPTION OF THE INVENTION

The disclosure provided herein describes methods and systems to determine, identify, classify, and/or generate one or more nucleic acid molecule features of one or more subjects that may differentiate, classify, and/or diagnose a health state of the one or more subjects and/or one or more groups of subjects. In some cases, the one or more nucleic acid molecule features may be derived, obtained, received, and/or determined from one or more nucleic acid molecules of one or more biological samples of a subject and/or a plurality of subjects. In some cases the one or more nucleic acid molecules may comprise one or more mammalian nucleic acid molecules, one or more non-mammalian nucleic acid molecules, or a combination thereof. In some instances, the one or more non-mammalian nucleic acid molecules may comprise one or more nucleic acid molecules from bacterial, fungi, or a combination thereof. In some cases, the health state of the one or more subjects, as described elsewhere herein, may comprise a cancerous health state, a non-cancerous disease health state, a healthy health state, or a combination thereof. In some instances, the cancerous health state may comprise an individual with cancer. In some cases, the cancer may comprise lung, breast, ovarian, gastro-intestinal, head and neck, liver, pancreas, prostate, skin, or any combination thereof cancer. In some cases, the lung cancer may comprise non-small cell lung cancer. In some cases, the cancerous health state may comprise a diagnosis of a cancer's stage (e.g., Stage I, Stage II, Stage II, etc.). In some cases the health state may comprise a spatial location (i.e., an anatomical location) of the cancer and/or disease within the subject or plurality of subjects. In some cases the biological samples may comprise a liquid biological sample, tissue biological sample, or a combination thereof. In some cases, the non-cancerous disease health state may comprise lung disease. In some instances, lung disease may comprise: carcinoid, hamartoma, granuloma, interstitial fibrosis, emphysema, bronchitis, chronic obstructive pulmonary disease, pneumonia, sarcoidosis, or any combination thereof. In some cases, the liquid biological sample may comprise a liquid biopsy. In some cases, the liquid biopsy may comprise plasma, serum, whole blood, urine, cerebral spinal fluid, saliva, sweat, tears, exhaled breath condensate, or any combination thereof. In some cases, the tissue biological sample may comprise a tissue biopsy of one or more regions, organs, and/or anatomical locations of a subject (e.g., lung, skin, liver, pancreas, brain, etc.).

In some cases, amplifying, enriching, filtering, and/or decontaminating the one or more nucleic acid molecules and/or one or more sequencing reads of the one or more nucleic acid molecules may provide better than expected results when the corresponding one or more enriched, filtered, and/or decontaminated nucleic acid molecule features determine, classify, identify, and/or diagnose a health state of one or more subjects with an accuracy of at least about 80%, at least about 85%, at least about 86%, at least about 87%, at least about 88%, at least about 89%, at least about 90%, at least about 92%, at least about 94%, at least about 96%, at least about 98%, or at least about 99%.

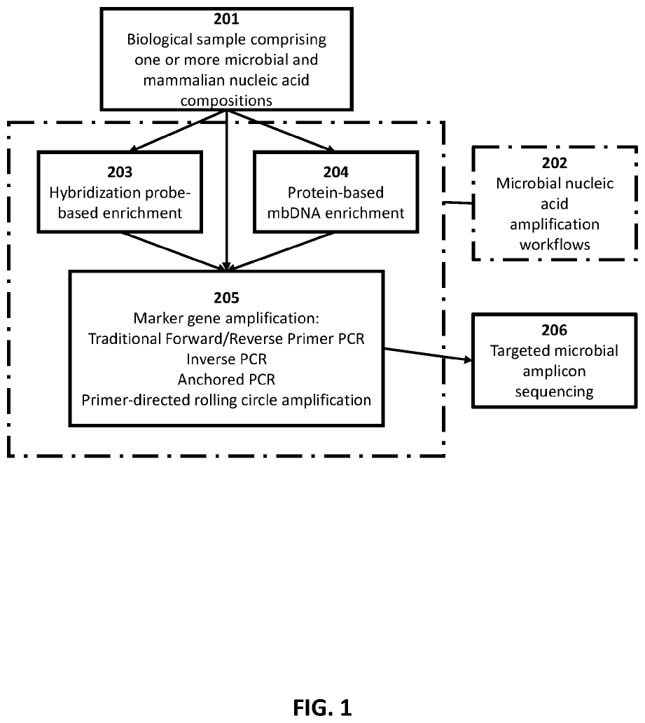

In some cases, the biological sample of the one or more subjects may comprise one or more microbial nucleic acid molecule compositions, one or more mammalian nucleic acid molecule compositions, or a combination thereof, 201, as seen in FIG. 1. In some cases, the one or more microbial nucleic acid molecules, one or more mammalian nucleic acid molecule compositions, or a combination thereof, may be enriched via a microbial nucleic acid enrichment and amplification workflow 202. In some cases, a biological sample comprising one or more microbial nucleic acid molecules and one or more mammalian nucleic acid molecules may be enriched by hybridization probe enrichment 203 and/or by protein-based microbial DNA enrichment 204. In some cases, hybridization based enrichment may comprise: combining the one or more mammalian nucleic acid molecules and the one or more non-mammalian nucleic acid molecule with the hybridization probes, wherein the hybridization probes may comprise a nucleic acid sequence complimentary to non-mammalian genomic features; incubating the hybridization probes, the one or more mammalian nucleic acid molecules, and the one or more on-mammalian nucleic acid molecules under conditions that promote nucleic acid molecule base pairing between target nucleic acid features of the one or more non-mammalian nucleic acid molecules and the hybridization probes; separating unbound hybridization probes and hybridized probes bound to the one or more non-mammalian nucleic acid molecules; and washing the hybridized probes bound to the one or more non-mammalian nucleic acid molecules, thereby generating one or more enriched non-mammalian nucleic acid molecules.

### Methods

In some instances, the disclosure provided herein describes a method of enriching one or more non-mammalian nucleic acid molecules (e.g., non-mammalian DNA). In some cases, enriching the one or more non-mammalian nucleic acid molecules may be enriched by protein-based non-mammalian (e.g., microbial) nucleic acid molecule enrichment 204. In some cases, the non-mammalian DNA enrichment may comprise: combining the one or more mammalian nucleic acid molecules and the one or more non-mammalian nucleic acid molecules with one or more recombinant CXXC-domain proteins to form a protein-DNA binding reaction; incubating the protein-DNA binding reaction under conditions that promote an interaction between the recombinant CXXC-domain proteins and non-methylated CpG motifs of the one or more mammalian nucleic acid molecules or the one or more non-mammalian nucleic acid molecules; separating unbound recombinant CXXC-domain proteins and recombinant CXXC-domain proteins bound to the non-methylated CpG nucleic acid fragments from the remainder of the protein-DNA binding reaction; and washing the recombinant CXXC-domain proteins bound to the non-methylated CpG nucleic acid fragments, thereby generating one or more enriched nucleic acid molecules for amplification.

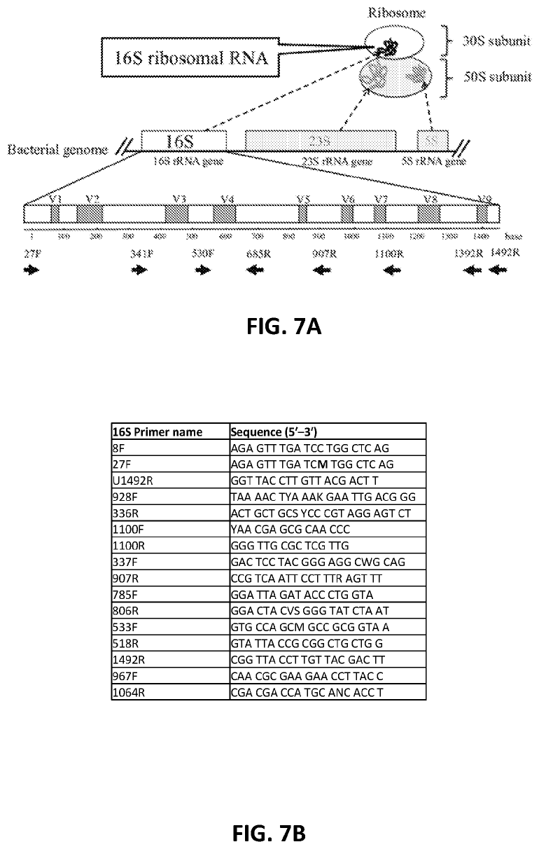

In some cases amplification may comprise marker gene amplification 205. In some cases the marker gene may comprise microbial phylogenetic marker genes or marker gene fragments thereof. In some instances, the microbial phylogenetic marker genes may comprise bacterial, fungal, or any combination thereof marker genes. In some cases, the microbial phylogenetic marker genes may comprise bacterial marker genes or marker gene fragments thereof. In some cases, the microbial phylogenetic marker genes may comprise fungal marker genes or marker gene fragments thereof. In some cases, the bacterial marker genes may comprise ribosomal RNA gene 5S, ribosomal RNA gene 16S, ribosomal RNA gene 23S, bacterial housekeeping genes dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, tsf, or any combination thereof. In some instances, the bacterial ribosomal RNA gene, as shown in FIG. 7A may comprise hypervariable regions (V1-V9) that may be utilized to differentiate and/or classify microbe taxonomy. In some cases, one or more forward and/or reverse primers, as shown in FIG. 7B, may be used to amplify 16S regions of the bacterial ribosomal RNA gene to differentiate phylogenetically diverse set of bacteria that may be used as a feature to differentiate, determine, and/or diagnose a health state of a subject and/or a group of subjects.

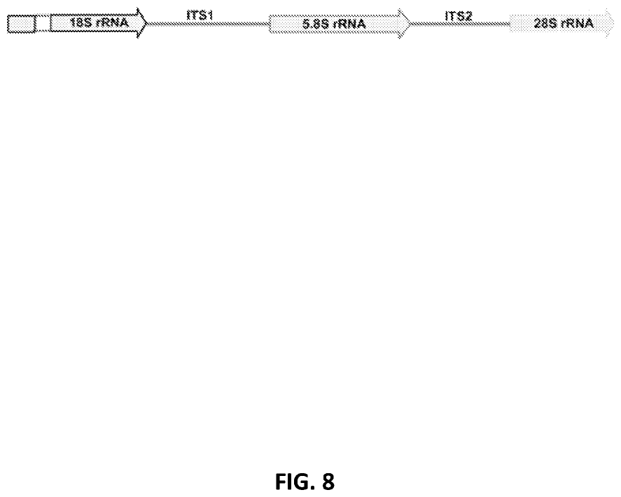

In some instances, the fungal marker genes may comprise: ribosomal RNA gene 18S, ribosomal RNA gene 5.8S, ribosomal RNA gene 28S, the internal transcribed spacer regions 1 and 2, or any combination thereof. In some cases, the internal transcribed spacer regions 1 and 2 (ITS1 and ITS2, respectively) are situated between small and large ribosomal RNA (rRNA) submits 18S rRNA, 5.8S rRNA, and 28S RNA, as shown in FIG. 8. In some cases, amplification, and sequencing, as described elsewhere herein, of the ITS1 and/or ITS2 region provide a genomic feature and/or label to detect and/or determine presence of one or more fungi in a biological sample of a subject and/or group of subjects. In some instances, the one or more fungi may provide a taxonomic feature that may differentiate, classify, and/or diagnose a health state of a subject and/or a plurality of subjects. In some cases, the ITS1 and/or ITS2 region. In some cases, amplification may be achieved and/or completed by performing polymerase chain reaction (PCR) or derivatives thereof. In some cases, the derivatives of polymerase chain reaction may comprise reverse primer PCR, inverse PCR, anchored PCR, primer-directed rolling circle amplification, or any combination thereof. In some instances, the polymerase chain reaction amplification may comprise blocking primers, marker gene primers, or a combination thereof that are configured to prevent amplification of one or more genomic features. In some cases, the one or more genomic features may comprise mitochondrial DNA genomic features.

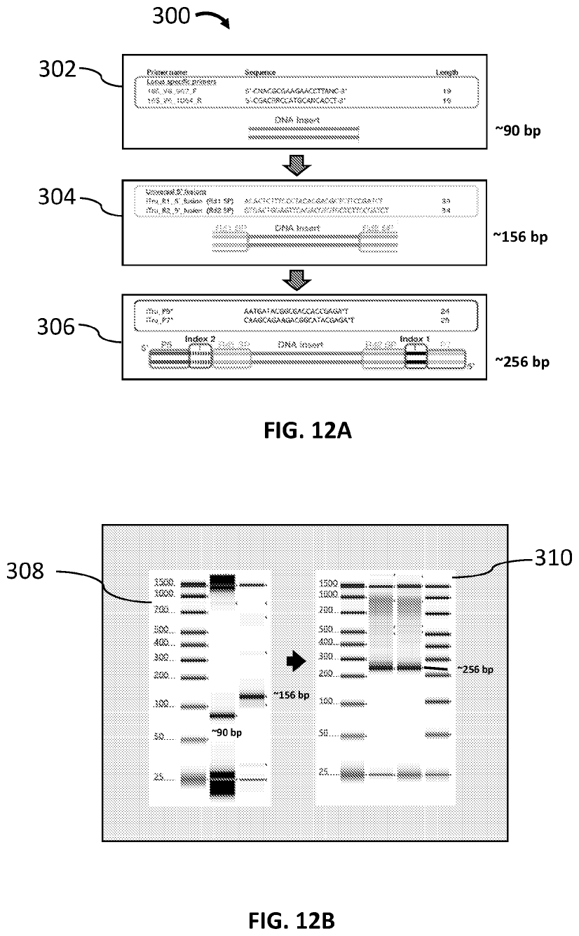

In some cases, the enriched and/or amplified one or more nucleic acid molecules may be prepared for sequencing through sequence library preparation 300, as shown in FIG. 12A. In some cases, the method may comprise: providing an amplified and/or enriched one or more nucleic acid 302; coupling barcoding index sequences 304; and coupling one or more adapter sequences to the barcoding index sequences 306. In some cases, the library of nucleic acid molecules may comprise a base pair length of about 256 bp. In some cases, the amplified and/or enriched one or more nucleic acid molecules may comprise a length of about 90 bp. In some instances, the amplified one or more nucleic acid molecule may comprise cell-free DNA of a plasma biological sample amplified with a V6 primer. As shown in the gel electrophoresis results of an example experiment shown FIG. 12B, the various lengths of the enriched and/or amplified nucleic acid molecule increases from before library preparation 308 to after library preparation 310.

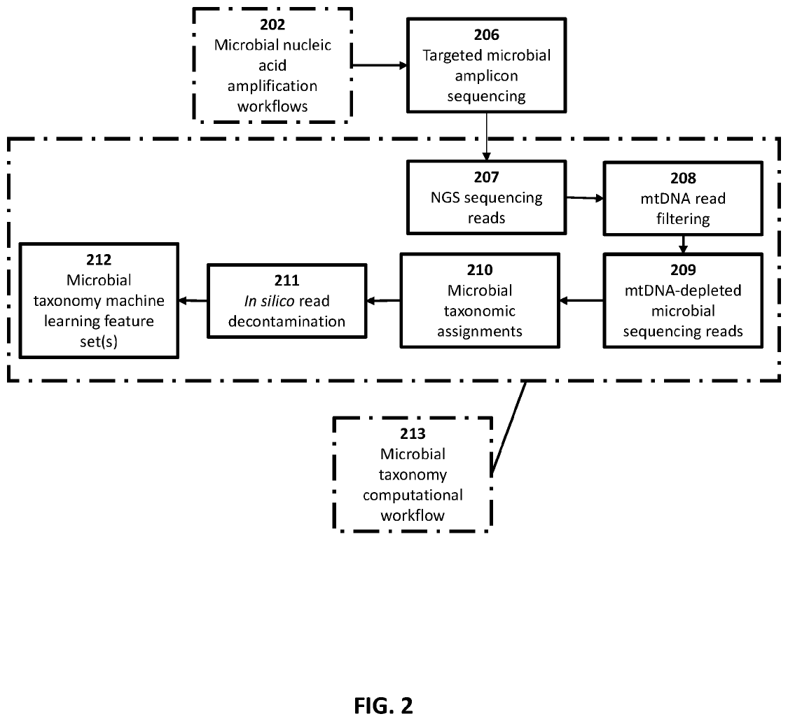

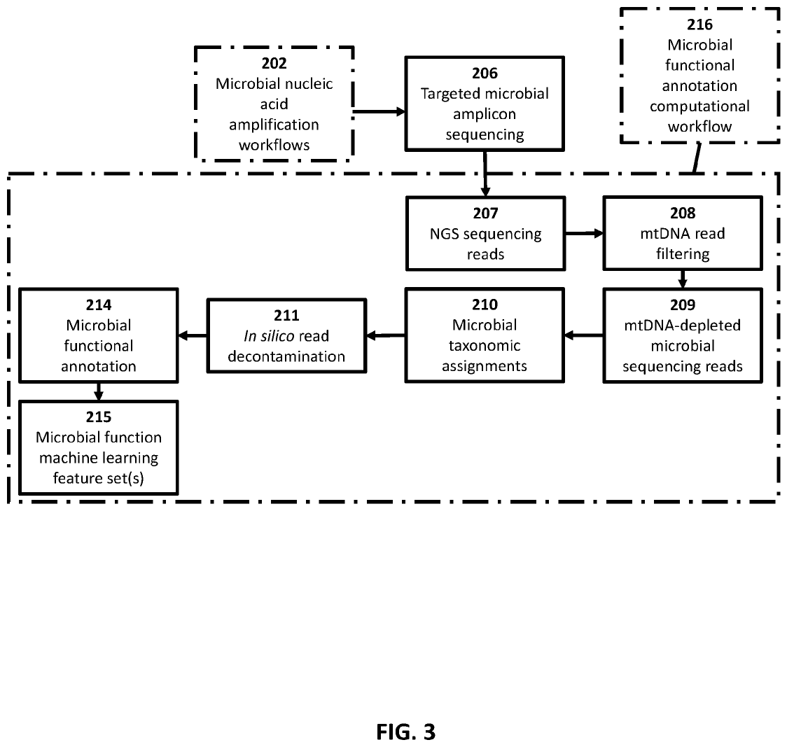

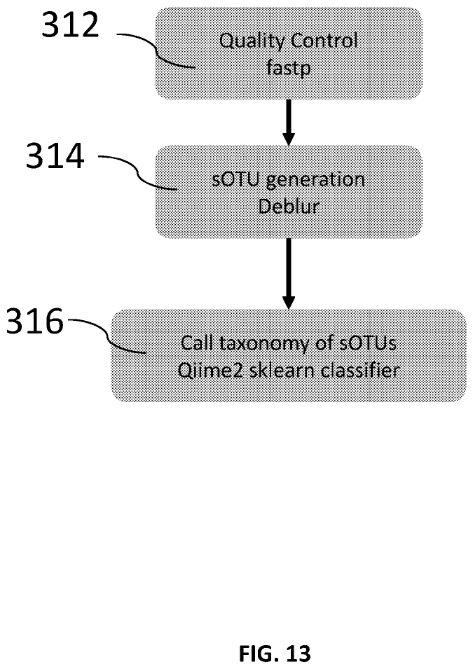

The resulting prepared library of the one or more enriched and/or amplified nucleic acid molecules composition(s) of mammalian and/or non-mammalian nucleic acid molecule may then be sequenced by targeted amplicon sequencing 206 methods e.g., targeted microbial amplicon sequencing may be used in a microbial taxonomy feature method 213 and/or a microbial functional feature method 216, as shown in FIGS. 2 and 3, respectively. In some cases, the targeted microbial amplicon sequencing may comprise microbial 16S amplicon sequencing. In some cases, the one or more sequencing reads generated by sequencing the one or more enriched and/or amplified nucleic acid molecule compositions may be pre-processed, as shown in FIG. 13. In some instances, the pre-processing may comprise: processing one or more sequencing reads of the enriched and/or amplified nucleic acid composition through fastp to remove adapter sequences and perform quality control to generate one or more processed sequencing reads 312; generating sub-operational taxonomy units from the processed one or more sequencing reads to perform quality 314; and querying the sub-operational taxonomy units against a genome database to assign one or more sub-operational unit taxonomies 316. In some cases, the quality control may comprise average read quality of about 30 or at least about 30. In some cases, the genome database may comprise 16S GreenGenes 13.8. In some cases, Qiime2's sklearn classifier may be used to assign sub-operational unit taxonomy. In some cases, sub-operational taxonomy units may be generated using Deblur, a denoising tool that models error profile of sequences based on quality scores, expected error rates, the observed frequency of each unique sequence, or a combination thereof. In some cases, the targeted microbial amplicon sequencing 206 may comprise shotgun sequencing, next generation sequencing 207, sequencing by synthesis, or a combination thereof. In some cases, the microbial taxonomy feature 213 and/or the microbial functional features may be part of a set of one or more nucleic acid molecule features, as described elsewhere herein. In some cases, the microbial taxonomy future method 213 may determine one or more microbial taxonomic assignments and associated microbial abundance of the enriched and/or amplified nucleic acid molecules. In some instances, the microbial functional feature method may determine one or more microbial functional pathways of the enriched and/or amplified nucleic acid molecules.

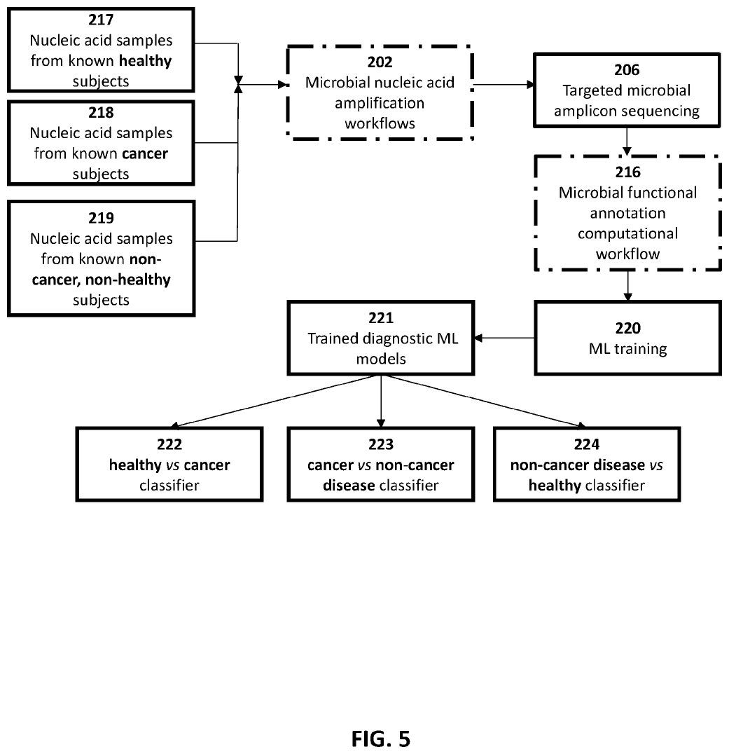

In some cases, the microbial functional feature method 216 (FIG. 3) may comprise: sequencing the enriched and/or amplified nucleic acid molecule library, e.g., using next generation sequencing, to generate a set of sequencing reads 207; filtering one or more nucleic acid molecule sequences (e.g., mitochondrial DNA) from the set sequencing of reads 208 thereby generating one or more mitochondrial DNA depleted sequencing reads 209; identifying one or more microbial taxonomic assignments of the one or more mitochondrial DNA depleted sequencing reads 210; decontaminating the one or more microbial taxonomic assignments 211; annotating and/or identify one or more microbial functional features of the one or more decontaminated microbial taxonomic sequencing reads 214; and outputting a feature set of the one or more identified and/or annotated microbial functional features 215. In some cases, the one or more microbial functional features 215 may be used in combination with a known health state of a subject (217, 218, 219) to train a predictive model (e.g., machine learning classifier), as shown in FIG. 5, described elsewhere herein. In some cases, the microbial functional features may comprise metagenomic functional features. In some cases, PICRUSt2 may determine and/or identify the one or more metagenomic functional features of the one or more decontaminated microbial taxonomic sequencing reads. In some cases, microbial taxonomy workflow may comprise mapping to determine microbial taxonomic assignments from the mitochondrial DNA depleted sequencing reads 210. In some cases, decontaminating may comprise in-silico decontamination. In some instances, decontaminating may remove one or more non-endogenous microbial sequencing reads, thereby generating one or more decontaminated microbial taxonomic assignments and associated quantity of sequencing reads from the one or more microbial taxonomic identities of the mitochondrial DNA-depleted non-mammalian sequencing reads.

In some cases, the microbial functional method 213 may comprise: sequencing the enriched and/or amplified nucleic acid molecule library, e.g., using next generation sequencing, to generate a set of sequencing reads 207; filtering one or more nucleic acid molecule sequences (e.g., mitochondrial DNA) from the set sequencing of reads 208 thereby generating one or more mitochondrial DNA depleted sequencing reads 209; identifying one or more microbial taxonomic assignments of the one or more mitochondrial DNA depleted sequencing reads 210; decontaminating the one or more microbial taxonomic assignments 211; and outputting one or more decontaminated microbial taxonomy features of the enriched and/or amplified nucleic acid molecule library 212. In some cases, the one or more microbial taxonomy features may be used in combination with a known health state of a subject (217, 218, 219) to train a predictive model (e.g., machine learning classifier), as shown in FIG. 5, described elsewhere herein. In some cases, microbial taxonomy workflow may comprise mapping to determine microbial taxonomic assignments from the mitochondrial DNA depleted sequencing reads 210. In some cases, mapping may comprise mapping the one or more mitochondrial DNA-depleted nucleic acid molecule sequencing reads against one or more microbial reference databases to determine microbial taxonomic identity of the mitochondrial DNA-depleted non-mammalian sequencing reads. In some cases, mapping may be performed by QIME2 or other supported versions thereof. In some cases, the one or more microbial reference databases may comprise: the bacterial 16S rRNA database Greengenes; the bacterial, fungal, and archaeal rRNA database SILVA; the eukaryotic nuclear ribosomal ITS region database UNITE; a custom database derived from publicly available and complete microbial genome sequences; or any combination thereof. In some cases, decontaminating may comprise in-silico decontamination. In some instances, decontaminating may remove one or more non-endogenous microbial sequencing reads, thereby generating one or more decontaminated microbial taxonomic assignments and associated quantity of sequencing reads from the one or more microbial taxonomic identities of the mitochondrial DNA-depleted non-mammalian sequencing reads.

In some cases, one or more nucleic acid molecule features may comprise genomic features of the one or more nucleic acid molecules. In some cases, the genomic features may comprise microbial phylogenetic marker genes or marker gene fragments thereof. In some instances, the microbial phylogenetic marker genes may comprise bacterial marker genes or marker gene fragments thereof. In some instances, the microbial phylogenetic marker genes may comprise fungal marker genes or marker gene fragments thereof. In some cases, the one or more nucleic acid molecule features may comprise a feature, a feature set and/or feature group of one or more non-mammalian nucleic acid molecules (e.g., microbial nucleic acid molecules), as described elsewhere herein.

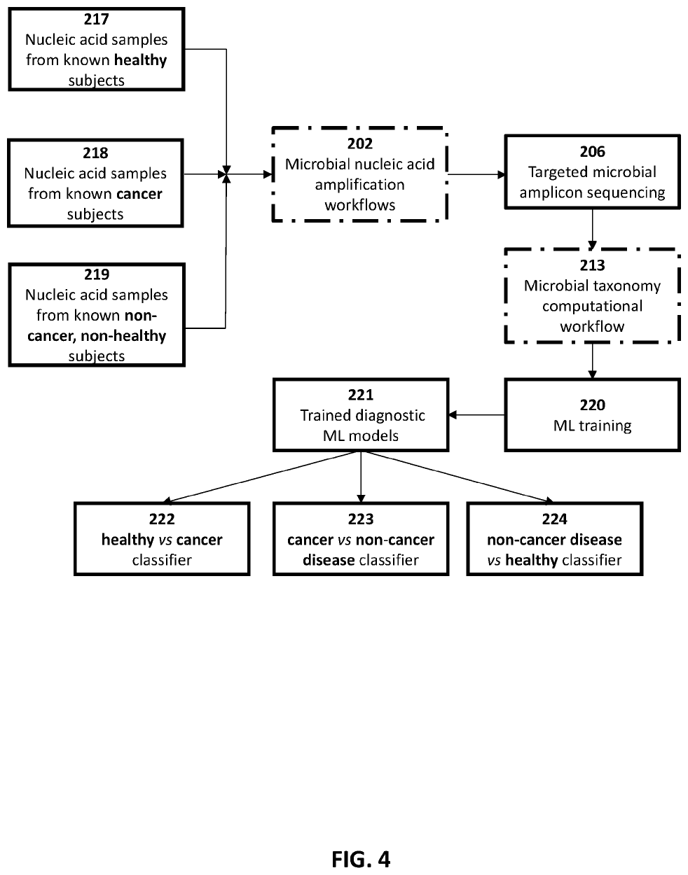

In some cases, the one or more nucleic acid molecule features may be used to train 220 one or more predictive models 221 (e.g., a machine learning classifier), as shown FIGS. 4 and 5, as described elsewhere herein. In some cases, the one or more nucleic acid molecule features (213, 216) may comprise microbial, bacterial, fungi, or a combination thereof taxonomy and/or functional classifications and/or characterization of the one or more nucleic acid molecules of a subject or a plurality of subjects' biological samples, as described elsewhere herein.

In some cases, predictive models (e.g., a machine learning classifier) may be trained with one or more nucleic acid molecule features of one or more nucleic acid molecules of a biological sample of subjects with a known health state of: healthy 217, non-cancerous disease 219, or cancerous 218. In some cases, the predictive model may be trained 220 with one or more microbial taxonomic features 213 and the associated health state of one or more subjects, as shown in FIG. 4. In some cases, the predictive model may be trained with one or more microbial functional features 216 and the associated health state of one or more subjects, as shown in FIG. 5. In some cases, the trained predictive models 221 may comprise one or more classifiers (222, 223, 224) that may differentiate, classify, and/or diagnose a health state of one or more subjects that were not included in the training of the predictive model. In some cases, the one or more classifiers may comprise a healthy vs cancer health state classifier 222, cancerous vs non-cancerous disease health classifier 223, a non-cancerous disease vs healthy classifier, or any combination thereof.

### Predictive Models

The methods and systems of the present disclosure may utilize or access external capabilities of artificial intelligence, predictive models, and/or machine learning trained on one or more nucleic acid molecule features that may classify, diagnose, and/or characterize a health state of a subject, a plurality of subjects and/or one or more groups of subjects. In some cases, the one or more nucleic acid molecule features (e.g., a microbial functional feature, a microbial taxonomic features, etc.), as described elsewhere herein may predict, classify, and/or identify a cancer and/or a non-cancerous disease of one or more subjects. In some cases, one or more nucleic acid molecule features may be used to train one or more predictive models, described elsewhere herein. These features may be used to accurately predict, classify, and/or characterize a health state e.g., cancer, non-cancerous diseases, disorders, or any combination thereof, of a subject, a plurality of subjects and/or one or more groups of subjects. Using such a predictive capability, health care providers (e.g., physicians) may be able to make informed, accurate risk-based decisions, thereby improving quality of care and monitoring provided to subjects with cancer, non-cancerous diseases, disorders, or any combination thereof.

The methods and systems of the present disclosure may analyze the presence and/or abundance of a microbes (e.g., abundance of microbes of a particular genus, taxonomy, microbial functional pathways). The presence and/or abundance of microbes may then be used to determine one or more nucleic acid molecule features e.g., non-mammalian nucleic acid molecule features that may predict cancer and/or non-cancerous diseases of one or more subjects. In some cases, the methods, and/or systems, described elsewhere herein, may train a predictive model with the one or more nucleic acid molecule features indicative of a health state e.g., cancer and/or a non-cancerous disease of a subject. In some cases, the trained predictive model may then be used to generate a likelihood (e.g., a prediction) of cancer and/or a non-cancerous disease of one or more subjects that differ from the one or more subjects utilized to train the predictive model. The trained predictive model may comprise an artificial intelligence-based model, such as a machine learning based classifier, configured to process one or more nucleic acid molecule features from the one or more nucleic acid molecules and/or enriched, filtered, and/or amplified one or more nucleic acid molecules, to generate the likelihood of the subject(s) having cancer, a non-cancerous disease, or a disorder. The model may be trained using abundance of microbial taxonomic features or microbial functional pathways from one or more cohorts of subjects, e.g., cancer subjects, subjects with non-cancerous diseases, subjects with no disease and no cancer, cancer subjects receiving a treatment for a cancer, subjects receiving treatment for a non-cancerous disease, or any combination thereof. In some cases, the predictive model may be trained to provide a treatment prediction to treat a cancer of one or more subjects that are not part of the training dataset of the predictive model. Such a predictive model may output a treatment recommendation for the one or more subjects that are not part of the training dataset when provided an input of the patient's presence and abundance of one or more microbes of a hybridization enriched biological sample.

The predictive model may comprise one or more predictive models. The model may comprise one or more machine learning algorithms. Examples of machine learning algorithms may include a support vector machine (SVM), a naïve Bayes classification, a random forest, a neural network (such as a deep neural network (DNN)), a recurrent neural network (RNN), a deep RNN, a long short-term memory (LSTM) recurrent neural network (RNN), a gated recurrent unit (GRU), a gradient boosting machine, a random forest, or other supervised learning algorithm or unsupervised machine learning, statistical, linear regression, k-nearest neighbors, k-means, decision tree, logistic regression, or any combination thereof. The model may be used for classification or regression. The model may likewise involve the estimation of ensemble models, comprised of multiple predictive models, and utilize techniques such as gradient boosting, for example in the construction of gradient-boosting decision trees. The model may be trained using one or more training datasets comprising one or more nucleic acid molecule features, subject data e.g., subject medical history, subject's family medical history, subject vitals (e.g., blood pressure, pulse, temperature, oxygen saturation), subject's known health state, or any combination thereof.

The predictive model may comprise any number of machine learning algorithms. In some embodiments, the random forest machine learning algorithm may be an ensemble of bagged decision trees. The ensemble may be at least about 1, 2, 3, 4, 5, 10, 20, 30, 40, 50, 60, 70, 80, 90, 100, 120, 140, 160, 180, 200, 250, 500, 1000 or more bagged decision trees. The ensemble may be at most about 1000, 500, 250, 200, 180, 160, 140, 120, 100, 90, 80, 70, 60, 50, 40, 30, 20, 10, 5, 4, 3, 2 or less bagged decision trees. The ensemble may be from about 1 to 1000, 1 to 500, 1 to 200, 1 to 100, or 1 to 10 bagged decision trees.

In some embodiments, the machine learning algorithms may have a variety of parameters. The variety of parameters may be, for example, learning rate, minibatch size, number of epochs to train for, momentum, learning weight decay, or neural network layers etc.

In some embodiments, the learning rate may be between about 0.00001 to 0.1.

In some embodiments, the minibatch size may be at between about 16 to 128.

In some embodiments, the neural network may comprise neural network layers. The neural network may have at least about 2 to 1000 or more neural network layers.

In some embodiments, the number of epochs to train for may be at least about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 500, 1000, 10000, or more.

In some embodiments, the momentum may be at least about 0.1, 0.2, 0.3, 0.4, 0.5, 0.6, 0.7, 0.8, 0.9 or more. In some embodiments, the momentum may be at most about 0.9, 0.8, 0.7, 0.6, 0.5, 0.4, 0.3, 0.2, 0.1, or less.

In some embodiments, learning weight decay may be at least about 0.00001, 0.0001, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, or more. In some embodiments, the learning weight decay may be at most about 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, 0.001, 0.0001, 0.00001, or less.

In some embodiments, the machine learning algorithm may use a loss function. The loss function may be, for example, regression losses, mean absolute error, mean bias error, hinge loss, Adam optimizer and/or cross entropy.

In some embodiments, the parameters of the machine learning algorithm may be adjusted with the aid of a human and/or computer system.

In some embodiments, the machine learning algorithm may prioritize certain features. The machine learning algorithm may prioritize features that may be more relevant for detecting cancer, non-cancerous disease, disorder, or any combination thereof. The feature may be more relevant for detecting cancer, non-cancerous disease, and/or disorders, if the feature is classified more often than another feature in determining cancer, non-cancerous disease, and/or disorders. In some cases, the features may be prioritized using a weighting system. In some cases, the features may be prioritized on probability statistics based on the frequency and/or quantity of occurrence of the feature. The machine learning algorithm may prioritize features with the aid of a human and/or computer system.

In some cases, the machine learning algorithm may prioritize certain features to reduce calculation costs, save processing power, save processing time, increase reliability, or decrease random access memory usage, etc.

Training datasets may be generated from, for example, one or more cohorts of subjects having common cancer, non-cancerous disease, or disorder diagnosis. Training datasets may comprise one or more nucleic acid molecule features in the form of abundance taxonomic assignment features of microbes present in the biological sample and/or microbial functional pathways features of the microbes present in the biological sample of one or more subjects. Features may comprise a corresponding cancer diagnosis of one or more subjects to microbial features. In some cases, features may comprise patient information such as patient age, patient medical history, other medical conditions, current or past medications, clinical risk scores, and time since the last observation. For example, a set of features collected from a given patient at a given time point may collectively serve as a signature, which may be indicative of a health state or status of the patient at the given time point.

Labels may comprise clinical outcomes such as, for example, a presence, absence, diagnosis, and/or prognosis of cancer, non-cancerous disease, disorder, or a combination thereof, in the subject (e.g., patient). Clinical outcomes may comprise treatment efficacy (e.g., whether a subject is a positive or a negative responder to a cancer and/or disease-based treatment).

Input features may be structured by aggregating the data into bins or alternatively using a one-hot encoding. Inputs may also include feature values or vectors derived from the previously mentioned inputs, such as cross-correlations.

Training datasets may be constructed from presence and/or abundance of one or more nucleic acid mole features of e.g., one or more microbial taxonomic features, one or more microbial functional pathways, or a combination thereof, identified and/or classified from the enriched and/or amplified nucleic acid molecules of a biological sample indicative of cancer, non-cancerous diseases, disorders, or any combination thereof.

The model may process the input features to generate output values comprising one or more classifications, one or more predictions, or a combination thereof. For example, such classifications or predictions may include a binary classification of a cancer or no cancer present; presence of a non-cancerous disease; presence of a disorder; or any combination thereof classifications of a subject. In some cases, the one or more predictive models and/or machine learning algorithms may classify subjects between a group of categorical labels (e.g., ‘no cancer, non-cancer disease and/or disorder’, ‘apparent cancer, non-cancer disease and/or disorder’, and ‘likely cancer, non-cancer disease and/or disorder’); a likelihood (e.g., relative likelihood or probability) of developing a particular cancer, non-cancerous disease, and/or disorder; a score indicative of a presence of cancer, non-cancer disease and/or disorder, a ‘risk factor’ for the likelihood of mortality of the patient, and a confidence interval for any numeric predictions. Various machine learning techniques may be cascaded such that the output of a machine learning technique may also be used as input features to subsequent layers or subsections of the model.

In order to train the model (e.g., by determining weights and correlations of the model) to generate real-time classifications or predictions, the model can be trained using training datasets and/or one or more training features, described elsewhere herein. Such datasets and/or features may be sufficiently large to generate statistically significant classifications or predictions. For example, datasets may comprise one or more nucleic acid molecule features derived from sequencing data from fungal, viral, archaeal, bacterial, or any combination thereof microbe presence and/or abundance in one or more subjects' biological samples.

Datasets may be split into subsets (e.g., discrete or overlapping), such as a training dataset, a development dataset, and a test dataset. For example, a dataset may be split into a training dataset comprising 80% of the dataset, a development dataset comprising 10% of the dataset, and a test dataset comprising 10% of the dataset. The training dataset may comprise about 10%, about 20%, about 30%, about 40%, about 50%, about 60%, about 70%, about 80%, or about 90% of the dataset. The development dataset may comprise about 10%, about 20%, about 30%, about 40%, about 50%, about 60%, about 70%, about 80%, or about 90% of the dataset. The test dataset may comprise about 10%, about 20%, about 30%, about 40%, about 50%, about 60%, about 70%, about 80%, or about 90% of the dataset. In some embodiments, leave one out cross validation may be employed. Training sets (e.g., training datasets) may be selected by random sampling of a set of data corresponding to one or more patient cohorts to ensure independence of sampling. Alternatively, training sets (e.g., training datasets) may be selected by proportionate sampling of a set of data corresponding to one or more patient cohorts to ensure independence of sampling.

To improve the accuracy of model predictions and reduce overfitting of the model, the datasets may be augmented to increase the number of samples within the training set. For example, data augmentation may comprise rearranging the order of observations in a training record. To accommodate datasets having missing observations, methods to impute missing data may be used, such as forward-filling, back-filling, linear interpolation, and multi-task Gaussian processes. Datasets may be filtered, or batch corrected to remove or mitigate confounding factors. For example, within a database, a subset of subjects may be excluded.

The model may comprise one or more neural networks, such as a neural network, a convolutional neural network (CNN), a deep neural network (DNN), a recurrent neural network (RNN), or a deep RNN. The recurrent neural network may comprise units which can be long short-term memory (LSTM) units or gated recurrent units (GRU). For example, the model may comprise an algorithm architecture comprising a neural network with a set of input features, as described elsewhere herein, e.g., one or more nucleic acid molecule features, vital measurements, subject medical history, subject demographics, or any combination thereof. Neural network techniques, such as dropout or regularization, may be used during training the model to prevent overfitting. The neural network may comprise a plurality of sub-networks, each of which is configured to generate a classification or prediction of a different type of output information, which may be combined to form an overall output of the neural network. The machine learning model may alternatively utilize statistical or related algorithms including random forest, classification and regression trees, support vector machines, discriminant analyses, regression techniques, as well as ensemble and gradient-boosted variations thereof.

When the model generates a classification or a prediction of cancer, non-cancerous disease, disorder, or a combination thereof, a notification (e.g., alert or alarm) may be generated and transmitted to a health care provider, such as a physician, nurse, or other member of the subject's treating team within a hospital. Notifications may be transmitted via an automated phone call, a short message service (SMS), multimedia message service (MMS) message, an e-mail, and/or an alert within a dashboard. The notification may comprise output information such as a prediction of cancer, non-cancerous disease, and/or disorder; a likelihood of the predicted cancer, non-cancerous disease and/or disorder; a time until an expected onset of the cancer, non-cancerous disease and/or disorder; a confidence interval of the likelihood or time, a recommended course of treatment for the cancer, non-cancerous disease and/or disorder, or any combination thereof information.

To validate the performance of the model, different performance metrics may be generated. For example, an area under the receiver-operating characteristic curve (AUROC) may be used to determine the diagnostic, prognostic, screening, or any combination thereof capability of the model. For example, the model may use classification thresholds which are adjustable, such that specificity and sensitivity are tunable, and the receiver-operating characteristic curve (ROC) can be used to identify the different operating points corresponding to different values of specificity and sensitivity.

In some cases, such as when datasets are not sufficiently large, cross-validation may be performed to assess the robustness of a model across different training and testing datasets.

To calculate performance metrics such as sensitivity, specificity, accuracy, positive predictive value (PPV), negative predictive value (NPV), area under the precision-recall curve (AUPR), AUROC, or similar, the following definitions may be used. A “false positive” may refer to an outcome in which a positive outcome or result has been incorrectly or prematurely generated (e.g., before the actual onset of, or without any onset of, the cancer, non-cancerous disease and/or disorder). A “true positive” may refer to an outcome in which positive outcome or result has been correctly generated, when the patient has the cancer, non-cancerous disease and/or disorder (e.g., the patient shows symptoms of the cancer, non-cancerous disease and/or disorder, or the patient's record indicates the cancer, non-cancerous disease and/or disorder). A “false negative” may refer to an outcome in which a negative outcome or result has been generated, but the patient has the cancer, non-cancerous disease and/or disorder (e.g., the patient shows symptoms of the cancer, non-cancerous disease and/or disorder, or the patient's record indicates the cancer, non-cancerous disease and/or disorder). A “true negative” may refer to an outcome in which a negative outcome or result has been generated (e.g., before the actual onset of, or without any onset of, the cancer, non-cancerous disease and/or disorder).

The model may be trained until certain pre-determined conditions for accuracy or performance are satisfied, such as having minimum desired values corresponding to diagnostic accuracy measures. For example, the diagnostic accuracy measure may correspond to prediction of a likelihood of occurrence of a cancer, non-cancerous disease and/or disorder in the subject. As another example, the diagnostic accuracy measure may correspond to prediction of a likelihood of deterioration or recurrence of a cancer, non-cancerous disease and/or disorder for which the subject has previously been treated. Examples of diagnostic accuracy measures may include sensitivity, specificity, positive predictive value (PPV), negative predictive value (NPV), accuracy, AUPR, and AUROC corresponding to the diagnostic accuracy of detecting or predicting a cancer, non-cancerous disease and/or disorder.

For example, such a pre-determined condition may be that the sensitivity of predicting the cancer, non-cancerous disease and/or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the specificity of predicting the cancer, non-cancerous disease and/or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the positive predictive value (PPV) of predicting the cancer, non-cancerous disease and/or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the negative predictive value (NPV) of predicting the cancer, non-cancerous disease and/or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the area under the curve (AUC) of a Receiver Operating Characteristic (ROC) curve (AUROC) of predicting the cancer, non-cancerous disease and/or disorder comprises a value of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

As another example, such a pre-determined condition may be that the area under the precision-recall curve (AUPR) of predicting the cancer, non-cancerous disease and/or disorder comprises a value of at least about 0.10, at least about 0.15, at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the trained model may be trained or configured to predict the cancer, non-cancerous disease and/or disorder with a sensitivity of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the cancer, non-cancerous disease and/or disorder with a specificity of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the cancer, non-cancerous disease and/or disorder with a positive predictive value (PPV) of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the cancer, non-cancerous disease and/or disorder with a negative predictive value (NPV) of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the cancer, non-cancerous disease and/or disorder with an area under the curve (AUC) of a Receiver Operating Characteristic (ROC) curve (AUROC) of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the trained model may be trained or configured to predict the cancer, non-cancerous disease and/or disorder with an area under the precision-recall curve (AUPR) of at least about 0.10, at least about 0.15, at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

The training data sets may be collected from training subjects (e.g., humans). Each training has a diagnostic status indicating that they have either been diagnosed with the biological condition or have not been diagnosed with the cancer, non-cancerous disease and/or disorder.

In some embodiments, the model is a neural network or a convolutional neural network. See, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference.

In some embodiments, independent component analysis (ICA) is used to de-dimensionalize the data, such as that described in Lee, T.-W. (1998): Independent component analysis: Theory and applications, Boston, Mass: Kluwer Academic Publishers, ISBN 0-7923-8261-7, and Hyvärinen, A.; Karhunen, J.; Oja, E. (2001): Independent Component Analysis, New York: Wiley, ISBN 978-0-471-40540-5, which is hereby incorporated by reference in its entirety.

In some embodiments, principal component analysis (PCA) is used to de-dimensionalize the data, such as that described in Jolliffe, I. T. (2002). Principal Component Analysis. Springer Series in Statistics. New York: Springer-Verlag. doi: 10.1007/b98835. ISBN 978-0-387-95442-4, which is hereby incorporated by reference in its entirety.

SVMs are described in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of “kernels,” which automatically realizes a non-linear mapping to a feature space. The hyper-plane found by the SVM in feature space corresponds to a non-linear decision boundary in the input space.

Decision trees are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York. pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety.

Clustering (e.g., unsupervised clustering model algorithms and supervised clustering model algorithms) is described on pages 211-256 of Duda and Hart, Pattern Classification and Scene Analysis, 1973, John Wiley & Sons, Inc., New York, (hereinafter “Duda 1973”) which is hereby incorporated by reference in its entirety. As described in Section 6.7 of Duda 1973, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined. Similarity measures are discussed in Section 6.7 of Duda 1973, where it is stated that one way to begin a clustering investigation is to define a distance function and to compute the matrix of distances between all pairs of samples in the training set. If distance is a good measure of similarity, then the distance between reference entities in the same cluster will be significantly less than the distance between the reference entities in different clusters. However, as stated on page 215 of Duda 1973, clustering does not require the use of a distance metric. For example, a nonmetric similarity function s (x, x′) can be used to compare two vectors x and x′. Conventionally, s (x, x′) is a symmetric function whose value is large when x and x′ are somehow “similar.” An example of a nonmetric similarity function s (x, x′) is provided on page 218 of Duda 1973. Once a method for measuring “similarity” or “dissimilarity” between points in a dataset has been selected, clustering requires a criterion function that measures the clustering quality of any partition of the data. Partitions of the data set that extremize the criterion function are used to cluster the data. See page 217 of Duda 1973. Criterion functions are discussed in Section 6.8 of Duda 1973. More recently, Duda et al., Pattern Classification, 2nd edition, John Wiley & Sons, Inc. New York, has been published. Pages 537-563 describe clustering in detail. More information on clustering techniques can be found in Kaufman and Rousseeuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, New Jersey, each of which is hereby incorporated by reference. Particular exemplary clustering techniques that can be used in the present disclosure include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering, where no preconceived notion of what clusters should form when the training set is clustered, are imposed.

Regression models, such as that of the multi-category logit models, are described in Agresti, An Introduction to Categorical Data Analysis, 1996, John Wiley & Sons, Inc., New York, Chapter 8, which is hereby incorporated by reference in its entirety. In some embodiments, the model makes use of a regression model disclosed in Hastie et al., 2001, The Elements of Statistical Learning, Springer-Verlag, New York, which is hereby incorporated by reference in its entirety. In some embodiments, gradient-boosting models are used toward, for example, the classification algorithms described herein; these gradient-boosting models are described in Boehmke, Bradley; Greenwell, Brandon (2019). “Gradient Boosting”. Hands-On Machine Learning with R. Chapman & Hall. pp. 221-245. ISBN 978-1-138-49568-5., which is hereby incorporated by reference in its entirety. In some embodiments, ensemble modeling techniques are used; these ensemble modeling techniques are described in the implementation of classification models herein and are described in Zhou Zhihua (2012). Ensemble Methods: Foundations and Algorithms. Chapman and Hall/CRC. ISBN 978-1-439-83003-1, which is hereby incorporated by reference in its entirety.

In some embodiments, the machine learning analysis is performed by a device executing one or more programs (e.g., one or more programs stored in the Non-Persistent Memory or in Persistent Memory) including instructions to perform the data analysis. In some embodiments, the data analysis is performed by a system comprising at least one processor (e.g., a processing core) and memory (e.g., one or more programs stored in Non-Persistent Memory or in the Persistent Memory) comprising instructions to perform the data analysis.

### Computer Systems

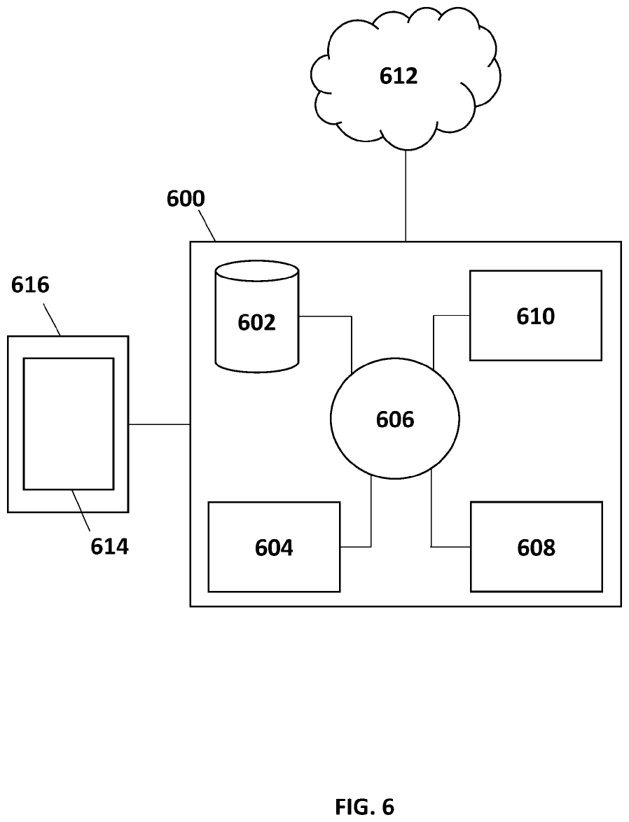

The present disclosure provides computer systems that are programmed to implement methods of the disclosure. FIG. 6 shows a computer system 600 that is programmed or otherwise configured to predict a health state of cancer, non-cancerous disease, or any combination thereof, of one or more subjects; train a predictive model, described elsewhere herein; generate a recommended therapeutic; or any combination thereof methods, described elsewhere herein. The computer system 600 can be an electronic device of a user or a computer system that is remotely located with respect to the electronic device. The electronic device can be a mobile electronic device.

The computer system 600 includes a central processing unit (CPU, also “processor” and “computer processor” herein) 606, which can be a single core or multi core processor, or a plurality of processors for parallel processing. The computer system 600 also includes memory or memory location 604 (e.g., random-access memory, read-only memory, flash memory), electronic storage unit 602 (e.g., hard disk), communication interface 608 (e.g., network adapter) for communicating with one or more other systems, and peripheral devices 610, such as cache, other memory, data storage and/or electronic display adapters. The memory 604, storage unit 602, interface 608 and peripheral devices 610 are in communication with the CPU 606 through a communication bus (solid lines), such as a motherboard. The storage unit 602 can be a data storage unit (or data repository) for storing data. The computer system 600 can be operatively coupled to a computer network (“network”) 612 with the aid of the communication interface 608. The network 612 can be the Internet, an internet and/or extranet, or an intranet and/or extranet that is in communication with the Internet. The network 612 in some cases is a telecommunication and/or data network. The network 612 can include one or more computer servers, which can enable distributed computing, such as cloud computing. The network 612, in some cases with the aid of the computer system 600, can implement a peer-to-peer network, which may enable devices coupled to the computer system 600 to behave as a client or a server.

The CPU 606 can execute a sequence of machine-readable instructions, which can be embodied in a program or software. The instructions may be stored in a memory location, such as the memory 604. The instructions can be directed to the CPU 606, which can subsequently program or otherwise configure the CPU 606 to implement methods of the present disclosure, described elsewhere herein. Examples of operations performed by the CPU 606 can include fetch, decode, execute, and writeback.

The CPU 606 can be part of a circuit, such as an integrated circuit. One or more other components of the system 600 can be included in the circuit. In some cases, the circuit is an application specific integrated circuit (ASIC).

The storage unit 602 can store files, such as drivers, libraries, and saved programs. The storage unit 602 can store user data, e.g., user preferences and user programs. The computer system 600 in some cases can include one or more additional data storage units that are external to the computer system 600, such as located on a remote server that is in communication with the computer system 600 through an intranet or the Internet.

The computer system 600 can communicate with one or more remote computer systems through the network 612. For instance, the computer system 600 can communicate with a remote computer system of a user. Examples of remote computer systems may include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 600 via the network 612.

Methods as described herein can be implemented by way of machine (e.g., computer processor) executable code stored on an electronic storage location of the computer system 600, such as, for example, on the memory 604 or electronic storage unit 602. The machine executable or machine-readable code can be provided in the form of software. During use, the code can be executed by the processor 606. In some cases, the code can be retrieved from the storage unit 602 and stored on the memory 604 for ready access by the processor 606. In some situations, the electronic storage unit 602 can be precluded, and machine-executable instructions are stored on memory 604.

The code can be pre-compiled and configured for use with a machine having a processer adapted to execute the code or can be compiled during runtime. The code can be supplied in a programming language that can be selected to enable the code to execute in a pre-compiled or as-compiled fashion.

In some embodiments, a system, as described elsewhere herein, may comprise a system for diagnosing a cancerous or non-cancerous health state of one or more subjects. In some cases, the system may comprise: (a) one or more processors; and (b) a non-transitory computer readable storage medium including software configured to cause said one or more processors to: (i) receive one or more subjects' one or more nucleic acid molecule sequencing reads of said one or more subjects' biological samples, wherein said one or more nucleic acid molecule sequencing reads comprise a sequence of an amplified one or more genomic features of one or more non-mammalian nucleic acid molecules; and (ii) output a diagnosis of a cancerous or non-cancerous health state of the one or more subjects at least as a result of providing the one or more non-mammalian nucleic acid sequencing reads' one or more genomic features as an input to a trained predictive model.

Aspects of the systems and methods provided herein, such as the computer system 600, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 600 can include or be in communication with an electronic display 616 that comprises a user interface (UI) 614 for providing, for example, a display for visualization of prediction results or an interface for training a predictive model. Examples of UI's include, without limitation, a graphical user interface (GUI) and web-based user interface.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Although the steps of the methods as described and claim herein show each of the methods or sets of operations in accordance with embodiments, a person of ordinary skill in the art will recognize many variations based on the teaching described herein. The steps may be completed in a different order. Steps may be added or omitted. Some of the steps may comprise sub-steps. Many of the steps may be repeated as often as beneficial.

One or more of the steps of each of the methods or sets of operations may be performed with circuitry as described herein, for example, one or more of the processor or logic circuitry such as programmable array logic for a field programmable gate array. The circuitry may be programmed to provide one or more of the steps of each of the methods or sets of operations and the program may comprise program instructions stored on a computer readable memory or programmed steps of the logic circuitry such as the programmable array logic or the field programmable gate array, for example.

### Example 1: 16S rDNA V6 Primer Amplification Efficiency and Specificity

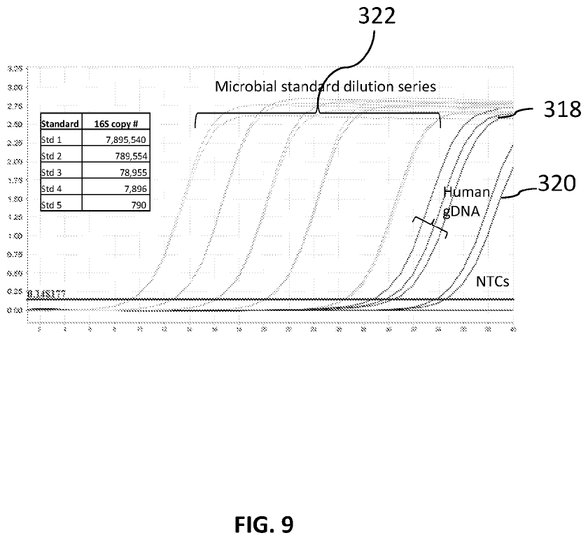

The efficiency of 16S rDNA V6 amplification primers e.g., 967F and 1064R were assessed and compared between human genome and microbial standard dilution series, as can be seen in FIG. 9. FIG. 9 shows polymerase chain reaction (PCR) cycle plotted against observed signal intensity of the PCR reaction product production for various compositions: human genome DNA 318, a microbial standard dilution series 322, and negative control 320 with no DNA present in the reaction. The microbial standard dilution series was comprised of 5 standard dilutions of 790, 7,896, 78,955, 789,554, and 7,895,540 16S copy numbers. From the PCR cycle plots, it can be seen that when only human genomic DNA is present 318, the V6 primers amplify human gDNA less efficiently when compared to the microbial standard dilution 322. These results suggest that the V6 primers selectively amplify microbial nucleic acid molecules thereby reducing noise or contaminant amplicons produced through the amplification of host mammalian mitochondrial DNA.

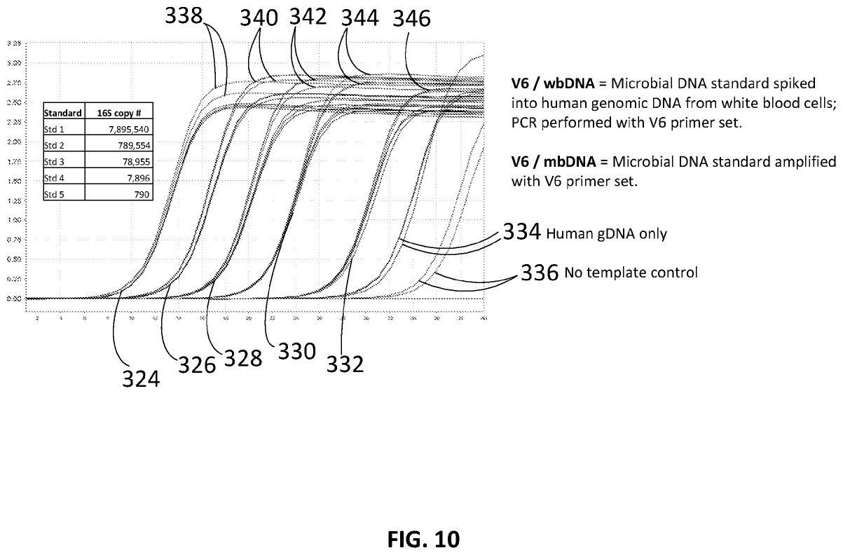

FIG. 10 shows an experiment performed to assess 16S rDNA V6 amplification primers (e.g., 967F, 1064R) efficiency amplified in the presence of human gDNA. Four groups were prepared for PCR amplification by V6 primers: microbial DNA standards spiked into human genomic DNA from white blood cells (V6/wbDNA) (324, 326, 328, 330, 332), microbial DNA standard (V6/mbDNA) (338, 340, 342, 344, 346), human gDNA only (334), and no template control (i.e., negative control) (336). The human gDNA content used in the reactions was fixed at 5 ng, corresponding to approximately 1,396 genome equivalents. On a mass basis, human gDNA was present in each experimental group at greater than or equal to 1000 times the amount of microbial DNA standard. The five microbial standard groups comprise 16S copy numbers of 790 (324, 338), 7,896 (326,340), 78,955 (328, 342), 789,554 (330, 344), and 7,895,540 (332, 346). From the results shown in FIG. 10, it can be understood that despite the amplification observed when only human gDNA is present in the PCR reaction, the human gDNA primer hybridization events do not impede specific amplification of microbial DNA when both DNA sources are mixed.

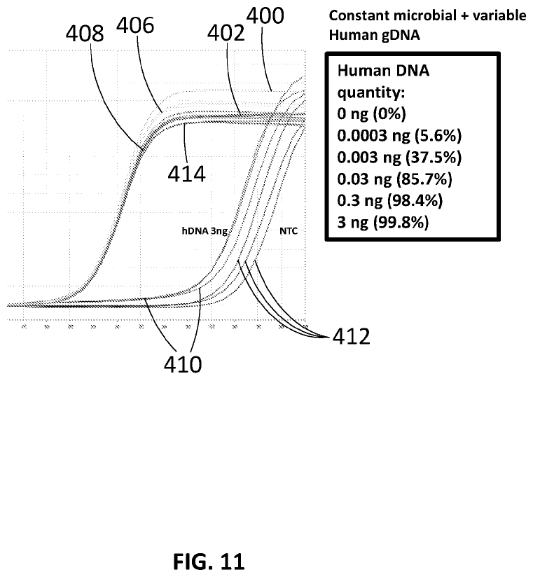

FIG. 11 shows an experiment performed to assess 16S rDNA V6 primer (e.g., 967F, 1064R) specificity of amplifying microbial DNA in the presence of human DNA. Six experimental groups with varying amounts of human genomic DNA: 3 ng (410), 0.3 ng (402), 0.03 ng (408), 0.003 ng (414), 0.0003 ng (406), and Ong (400) were prepared and amplified in the presence of 5 pg microbial DNA standard (7,895 genome equivalents). Additionally a no template control group (412) was also utilized as a negative control. From the PCR cycle plot shown in FIG. 37, it can be seen that at all levels of human gDNA spoked, microbial gDNA was preferentially amplified. In the absence of microbial DNA, the human DNA only control “hDNA 3 ng” curves amplify when reduced efficiency, yielding a Cp of 31. These results indicated that the V6 primers are highly specific and are not impeded by a significant foreground of human DNA since the PCR amplification curves are overlaid.

### Example 2: Amplicon Sequencing Blank Filtering

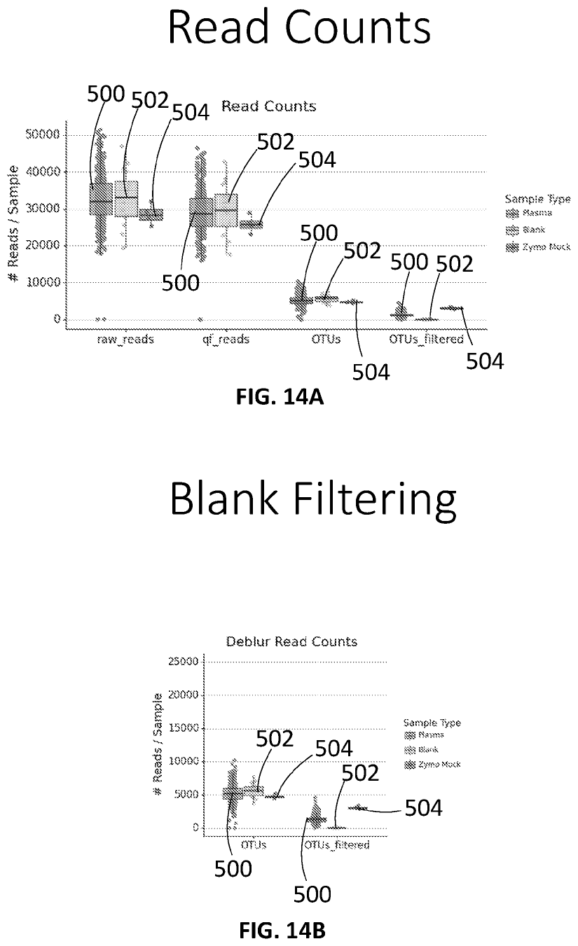

Amplicon sequencing read counts at varying steps of the workflow and/or methods described herein is shown in FIGS. 14A-14B. FIG. 14B shows a zoomed in view of the “OTUs” and “OTUs_filtered” sequencing reads/sample shown in FIG. 14A. Experimental groups included plasma (500), a blank negative control (502), and an industry Zymo commercially available sample of DNA microbes mixed at defined concentrations (504). The read number per sample plot shows read number/sample for various points of nucleic acid molecule sequencing reads as described elsewhere herein: “raw_reads” which are the total reads/sample prior to quality filter or taxonomic assignment, “qf_reads” are sequencing reads remaining after quality filtering steps to remove PCR duplications, “OTUs” correspond to the number of reads per sample that correspond to sub-operational taxonomy units identified via Deblur processing, and “OTUs_filtered” correspond to sOTUs remaining after subtraction of the sOTUs present in the DNA extraction blank controls (i.e., “Blank”). Features with an abundance of at least 10 within the whole dataset were retained for further downstream processing.

From the results shown in FIGS. 14A-14B, after accounting for contamination present in the DNA extraction process itself from subtracting sequencing reads from the blank control, there are thousands of decontaminated reads to process further to determine one or more nucleic acid features for classifying and/or identifying a health state of one or more subjects, as described elsewhere herein. 75.75% of total abundance was removed from the plasma samples post-blank filtering that translates to 12,509 microbes removed from a total of 30,689 microbes (40.76% of microbes removed). Accordingly, what is detected and analyzed by the methods and systems, described elsewhere herein, is not DNA contamination and rather is nucleic acid molecule signal present in a biological sample of a subject.

### Example 3: Machine Learning Classifier Performance

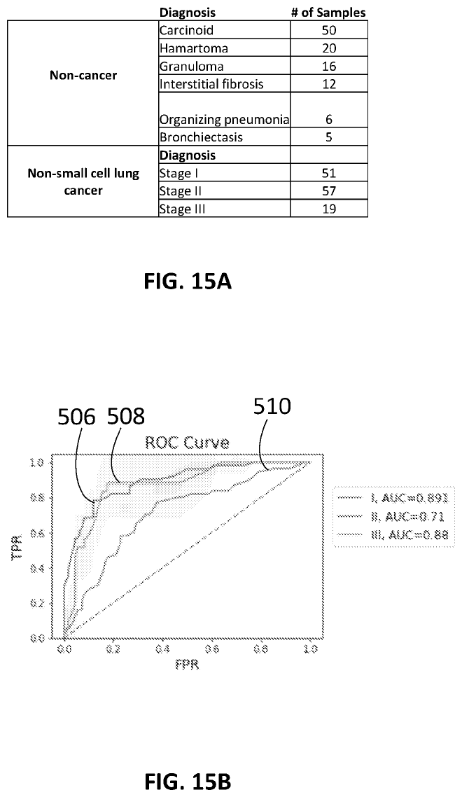

A machine learning classifier was trained with 16S amplicon sequences of one or more subjects (e.g., the V6 hypervariable region) with known health state labels i.e., a specific non-cancerous disease label and/or a stage of non-small cell lung cancer. The distribution of the number of subjects and/or samples of the various labeled health state is shown in FIG. 15A. Plasma from all subjects of each group was obtained and amplified with V6 16S primers follow by next generation sequencing, as described elsewhere herein. Sequencing reads were then decontaminated processed to identify one or more microbial taxonomy features. The microbial taxonomy features included abundances of the identified microbial taxonomy collapsed to a genus level. Associated read counts for microbial taxonomy features were then used to train three random forest machine learning model with 5-fold Cross Validation. The three random forest machine learning models included classifiers to classify and/or characterize cancer health states of a Stage I, Stage II, and Stage III. Performance receiver operating characteristic curves for the classifiers and associated area under the curve (AUC), namely 0.891 for stage I, 0.71 for stage II, and 0.88 for stage III cancer, are shown in FIG. 15B.

Three separate machine learning classifiers for stage I (506), stage II (510), and stage III (508) cancer health state classification and/or identification were generated using shotgun sequencing and Web of Life (WoL) genus relative abundance of the sequencing reads. The associated AUC for the machine learning classifiers trained with the shotgun sequencing and WOL abundances was 0.837 for stage I cancer, 0.66 for stage II cancer, and 0.871 for stage III cancer.

From these results it can be seen that V6 16S amplification primers provide one or more an enriched and/or amplified nucleic acid molecules may be used to develop one or more microbial taxonomic features that provide high accuracy in differentiating stage I, stage II, and stage III cancers.

What is claimed:

1. A method of generating a genomic feature set for differentiating a cancer and non-cancer health state of one or more subjects, the method comprising:
(a) providing one or more subjects' one or more nucleic acids molecules and corresponding health states;
(b) amplifying one or more genomic features of one or more non-mammalian nucleic acid molecules of said one or more nucleic acids molecules, thereby generating an amplified one or more genomic features;
(c) sequencing said amplified one or more genomic features to generate one or more non-mammalian sequencing reads; and
(d) generating a feature set configured to differentiate a cancer and non-cancer health state by combining said one or more genomic feature abundances of said one or more non-mammalian sequencing reads and said health state of said one or more subjects.

2. The method of claim 1, wherein said genomic features comprise microbial phylogenetic marker genes or marker gene fragments thereof.

3. The method of claim 2, wherein said microbial phylogenetic marker genes comprise bacterial marker genes or marker gene fragments thereof.

4. The method of claim 2, wherein said microbial phylogenetic marker genes comprise fungal marker genes or marker gene fragments thereof.

5. The method of claim 3, wherein said bacterial marker genes comprise: ribosomal RNA gene 5S, ribosomal RNA gene 16S, ribosomal RNA gene 23S, bacterial housekeeping genes dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, tsf, or any combination thereof.

6. The method of claim 4, wherein said fungal marker genes comprise: ribosomal RNA gene 18S, ribosomal RNA gene 5.8S, ribosomal RNA gene 28S, the internal transcribed spacer regions 1 and 2, or any combination thereof.

7. The method of claim 2, wherein microbial phylogenetic marker genes comprise bacterial, fungal, or any combination thereof marker genes.

8. The method of claim 1, wherein amplifying comprises performing a polymerase chain reaction or derivatives thereof.

9. The method of claim 8, wherein said derivatives thereof comprise inverse PCR, anchored PCR, primer-directed rolling circle amplification, or any combination thereof.

10. The method of claim 8, wherein said polymerase chain reaction comprises blocking primers, marker gene primers, or any combination thereof, configured to prevent amplification of one or more genomic features.

11. The method of claim 10, wherein said one or more genomic features comprise mitochondrial DNA genomic features.

12. The method of claim 1, comprising enriching said one or more nucleic acids molecules.

13. The method of claim 12, wherein said one or more nucleic acids molecules comprise mammalian, non-mammalian, or any combination thereof nucleic acids molecules.

14. The method of claim 13, wherein said one or more mammalian nucleic acid molecules comprise DNA, RNA, cell-free DNA, cell-free RNA, exosomal DNA, exosomal RNA, or any combination thereof.

15. The method of claim 13, wherein said one or more non-mammalian nucleic acid molecules comprise microbial DNA, microbial RNA, microbial cell free DNA, microbial cell free RNA, or any combination thereof.

16. The method of claim 13, wherein said enriching comprises:
(a) combining said one or more mammalian nucleic acid molecules and said non-mammalian nucleic acid molecules with hybridization probes, wherein said hybridization probes comprise a nucleic acid sequence complementarity to non-mammalian genomic features;
(b) incubating said hybridization probes, said one or more mammalian nucleic acid molecules, and said one or more non-mammalian nucleic acids under conditions that promote nucleic acid base pairing between target nucleic acid features of said one or more non-mammalian nucleic acid molecules and said hybridization probes;
(c) separating unbound hybridization probes and hybridized probes bound to said one or more non-mammalian nucleic acid molecules; and
(d) washing said hybridized probes bound to said one or more non-mammalian nucleic acid molecules, thereby generating one or more enriched non-mammalian nucleic acid molecules.

17. The method of claim 16, wherein washing is configured to remove non-specifically associated nucleic acids and other reaction components.

18. The method of claim 13, wherein enrichment of said one or more nucleic acid molecules comprises non-mammalian DNA enrichment.

19. The method of claim 18, wherein said non-mammalian DNA enrichment comprises:
(a) combining said one or more mammalian nucleic acid molecules and said one or more non-mammalian nucleic acid molecules with one or more recombinant CXXC-domain proteins to form a protein-DNA binding reaction;
(b) incubating said protein-DNA binding reaction under conditions that promote an interaction between said recombinant CXXC-domain proteins and non-methylated CpG motifs of said one or more mammalian nucleic acid molecules or said one or more non-mammalian nucleic acid molecules;
(c) separating unbound recombinant CXXC-domain proteins and recombinant CXXC-domain proteins bound to said non-methylated CpG nucleic acid fragments from the remainder of the protein-DNA binding reaction; and
(d) washing said recombinant CXXC-domain proteins bound to said non-methylated CpG nucleic acid fragments, thereby generating one or more enriched nucleic acids for amplification.

20. The method of claim 19, wherein washing is configured to remove non-specifically associated nucleic acid molecules and a remainder of said protein-DNA binding reaction components.

21. The method of claim 1, wherein said one or more nucleic acid molecules are derived from one or more biological samples of said one or more subjects.

22. The method of claim 21, wherein said one or more biological samples comprise a tissue, liquid, or any combination thereof biopsy sample.

23. The method of claim 1, wherein said one or more subjects comprise human, non-human mammal, or any combination thereof subjects.

24. The method of claim 1, comprising filtering said one or more non-mammalian sequencing reads.

25. The method of claim 24, wherein filtering comprises filtering said one or more non-mammalian sequencing reads to produce one or more mitochondrial DNA-depleted non-mammalian sequencing reads.

26. The method of claim 25, wherein filtering comprises mapping said one or more mitochondrial DNA-depleted non-mammalian sequencing reads against one or more microbial reference databases to determine microbial taxonomic identity of said one or more mitochondrial DNA-depleted non-mammalian sequencing reads.

27. The method of claim 26, comprising decontaminating said one or more mitochondrial DNA-depleted non-mammalian sequencing reads.

28. The method of claim 27, wherein decontaminating comprises in-silico decontamination.

29. The method of claim 27, wherein decontaminating is configured to remove non-endogenous microbial sequencing reads, thereby generating decontaminated microbial taxonomic assignments and associated quantity of sequencing reads.

30. The method of claim 26, wherein mapping is performed with QIIME2 or other supported versions thereof.

31. The method of claim 26, wherein said one or more microbial reference databases comprise the bacterial 16S rRNA database Greengenes; the bacterial, fungal and archaeal rRNA database SILVA; the eukaryotic nuclear ribosomal ITS region database UNITE; a custom database derived from publicly available and complete microbial genome sequences; or any combination thereof.

32. The method of claim 1, wherein said one or more genomic feature abundances of said one or more non-mammalian sequencing reads comprise microbial functional gene, biochemical pathway, or any combination thereof abundances.

33. The method of claim 29, comprising predicting metagenomic functional content of said decontaminated microbial taxonomic assignments, thereby producing one or more functional abundances.

34. The method of claim 33, wherein predicting said metagenomic functional content is performed by PICRUSt2.

35. The method of claim 22, wherein said liquid biopsy sample comprises: plasma, serum, whole blood, urine, cerebral spinal fluid, saliva, sweat, tears, exhaled breath condensate, or any combination thereof.

36. The method of claim 1, wherein said cancer comprises lung, breast, ovarian, gastro-intestinal, head and neck, liver, pancreas, prostate, skin, or any combination thereof cancers.

37. The method of claim 36, wherein said lung cancer comprises non-small cell lung cancer.

38. The method of claim 1, where said non-cancer health state comprises healthy, disease, or any combination thereof.

39. The method of claim 38, wherein said disease state comprises lung disease, wherein said lung disease comprises: carcinoid, hamartoma, granuloma, interstitial fibrosis, emphysema, bronchitis, chronic obstructive pulmonary disease, pneumonia, sarcoidosis, or any combination thereof.

40. The method of claim 1, wherein said cancer comprises a cancer of stage I, II, or III.

41. The method of claim 1, comprising generating a trained predictive model, wherein said trained predictive model is trained with said feature set and said health state of said one or more subjects.

42. The method of claim 41, wherein said trained predictive model comprises a machine learning model, one or more machine learning models, an ensemble of machine learning models, or any combination thereof.

43. The method of claim 41, wherein said trained predictive model comprises a regularized machine learning model.

44. The method of claim 42, wherein said machine learning model comprises a machine learning classifier.

45. The method of claim 42, wherein said machine learning model comprises a gradient boosting machine, neural network, support vector machine, k-means, classification trees, random forest, regression, or any combination thereof.

46. A method of diagnosing a cancer of one or more subjects, the method comprising:
(a) providing one or more subjects' one or more nucleic acid molecules;
(b) amplifying one or more genomic features of one or more non-mammalian nucleic acid molecules of said one or more nucleic acid molecules, thereby generating an amplified one or more genomic features;
(c) sequencing said amplified one or more genomic features to generate one or more non-mammalian sequencing reads; and
(d) outputting a diagnosis of a cancer or non-cancer health state of said one or more subjects at least as a result of providing said one or more genomic features as an input to a trained predictive model.

47. The method of claim 46, wherein said non-mammalian nucleic acid molecules comprise microbial nucleic acids.

48. The method of claim 46, wherein said one or more subjects comprise human, non-human mammal, or any combination thereof subjects.

49. The method of claim 46, wherein said one or more nucleic acid molecules comprise a total population of DNA, RNA, cell-free DNA, cell-free RNA, exosomal DNA, exosomal RNA, cell-free microbial DNA, cell-free microbial RNA, or any combination thereof.

50. The method of claim 46, wherein said one or more genomic features comprise microbial phylogenetic marker genes or marker gene fragments thereof.

51. The method of claim 50, wherein said microbial phylogenetic marker genes comprise bacterial marker genes or marker gene fragments thereof.

52. The method of claim 50, wherein said microbial phylogenetic marker genes comprise fungal marker genes or marker gene fragments thereof.

53. The method of claim 51, wherein said bacterial marker genes comprise: the ribosomal RNA gene 5S, ribosomal RNA gene 16S, ribosomal RNA gene 23S, bacterial housekeeping genes dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, tsf, or any combination thereof.

54. The method of claim 52, wherein said fungal marker genes may comprise: ribosomal RNA gene 18S, ribosomal RNA gene 5.8S, ribosomal RNA gene 28S, the internal transcribed spacer regions 1 and 2, or any combination thereof.

55. The method of claim 50, wherein said microbial phylogenetic marker genes comprise bacterial, fungal, or any combination thereof marker genes.

56. The method of claim 46, wherein amplifying comprises performing a polymerase chain reaction or derivatives thereof.

57. The method of claim 56, wherein said derivatives thereof comprise inverse PCR, anchored PCR, primer-directed rolling circle amplification, or any combination thereof.

58. The method of claim 56, wherein said polymerase chain reaction comprises blocking primers, marker gene primers, or any combination thereof configured to prevent amplification of one or more genomic features.

59. The method of claim 58, wherein said one or more genomic features comprise mitochondrial DNA genomic features.

60. The method of claim 46, comprising enriching said one or more nucleic acid molecules.

61. The method of claim 60, wherein said one or more nucleic acid molecules comprise one or more mammalian, non-mammalian, or any combination thereof nucleic acid molecules.

62. The method of claim 61, wherein enriching comprises:
(a) combining said one or more mammalian nucleic acid molecules and said one or more non-mammalian nucleic acid molecules with hybridization probes, wherein said hybridization probes comprise a nucleic acid sequence complementarity to non-mammalian genomic features;
(b) incubating said hybridization probes and said one or more mammalian nucleic acid molecules and said one or more non-mammalian nucleic acid molecules under conditions that promote nucleic acid base pairing between target nucleic acid features of said one or more non-mammalian nucleic acid molecules and said hybridization probes;
(c) separating unbound hybridization probes and hybridized probes bound to said one or more non-mammalian nucleic acid molecules; and
(d) washing said hybridized probes bound to said one or more non-mammalian nucleic acid molecules, thereby generating one or more enriched non-mammalian nucleic acid molecules.

63. The method of claim 62, wherein washing is configured to remove non-specifically associated nucleic acid molecules and other reaction components.

64. The method of claim 61, wherein enrichment of said one or more nucleic acid molecules comprises non-mammalian DNA enrichment.

65. The method of claim 64, wherein said non-mammalian DNA enrichment comprises:
(a) combining said one or more mammalian nucleic acid molecules and said one or more non-mammalian nucleic acid molecules with one or more recombinant CXXC-domain proteins to form a protein-DNA binding reaction;
(b) incubating said protein-DNA binding reaction under conditions that promote an interaction between said recombinant CXXC-domain proteins and non-methylated CpG motifs of said one or more mammalian nucleic acid molecules or said one or more non-mammalian nucleic acids;
(c) separating unbound recombinant CXXC-domain proteins and recombinant CXXC-domain proteins bound to said non-methylated CpG nucleic acid molecule fragments from a remainder of the protein-DNA binding reaction;
(d) washing said recombinant CXXC-domain proteins bound to said non-methylated CpG nucleic acid molecule fragments, thereby generating one or more enriched nucleic acids for amplification.

66. The method of claim 65, wherein said recombinant CXXC-domain proteins comprise: recombinant zinc finger CXXC domain-containing proteins KDM2A, KDM2A, KDM2B, FBXL19, CFP1, DNMT1, MLL1, MLL2, MDB1, TET1, TET3, IDAX, CXXC5, CGBP, the recombinant CXXC domains derived therefrom, or any combination thereof.

67. The method of claim 65, wherein washing is configured to remove non-specifically associated nucleic acid molecules and said remainder of said protein-DNA binding reaction components.

68. The method of claim 46, wherein said one or more nucleic acid molecules are derived from one or more biological samples of said one or more subjects.

69. The method of claim 68, wherein said one or more biological samples comprise a tissue, liquid, or any combination thereof biopsy sample.

70. The method of claim 61, wherein said one or more mammalian nucleic acid molecules comprise DNA, RNA, cell-free DNA, cell-free RNA, exosomal DNA, exosomal RNA, or any combination thereof.

71. The method of claim 61, wherein said one or more non-mammalian nucleic acid molecules comprise microbial DNA, microbial RNA, microbial cell free DNA, microbial cell free RNA, or any combination thereof.

72. The method of claim 61, comprising filtering said one or more non-mammalian sequencing reads.

73. The method of claim 72, wherein filtering comprises filtering said one or more non-mammalian sequencing reads to produce one or more mitochondrial DNA-depleted non-mammalian sequencing reads.

74. The method of claim 73 wherein filtering comprises mapping said one or more mitochondrial DNA-depleted non-mammalian sequencing reads against one or more microbial reference databases to determine microbial taxonomic identity of said one or more mitochondrial DNA-depleted non-mammalian sequencing reads.

75. The method of claim 74, comprising decontaminating said one or more mitochondrial DNA-depleted non-mammalian sequencing reads.

76. The method of claim 75, wherein decontaminating comprises in-silico decontamination.

77. The method of claim 75, wherein decontaminating is configured to remove non-endogenous microbial sequencing reads, thereby generating decontaminated microbial taxonomic assignments and associated quantity of sequencing reads.

78. The method of claim 74, wherein mapping is performed with QIIME2 or other supported versions thereof.

79. The method of claim 74, wherein said one or more microbial reference databases comprise the bacterial 16S rRNA database Greengenes; the bacterial, fungal and archaeal rRNA database SILVA; the eukaryotic nuclear ribosomal ITS region database UNITE; a custom database derived from publicly available and complete microbial genome sequences; or any combination thereof.

80. The method of claim 46, wherein said one or more genomic features comprise an abundance of said one or more non-mammalian sequencing reads' microbial functional genes, biochemical pathways, or any combination thereof abundances.

81. The method of claim 77, comprising predicting metagenomic functional content of said decontaminated microbial taxonomic assignments, thereby producing one or more functional abundances.

82. The method of claim 81, wherein predicting said metagenomic functional content is performed by PICRUSt2.

83. The method of claim 69, wherein said liquid biopsy sample comprises: plasma, serum, whole blood, urine, cerebral spinal fluid, saliva, sweat, tears, exhaled breath condensate, or any combination thereof.

84. The method of claim 46, wherein said cancer health state comprises lung, breast, ovarian, gastro-intestinal, head and neck, liver, pancreas, prostate, skin, or any combination thereof cancers.

85. The method of claim 84, wherein said lung cancer comprises non-small cell lung cancer.

86. The method of claim 46, where said non-cancer state comprises healthy, disease, or any combination thereof non-cancer states.

87. The method of claim 86, wherein said disease comprises lung disease, wherein said lung disease comprises: carcinoid, hamartoma, granuloma, interstitial fibrosis, emphysema, bronchitis, chronic obstructive pulmonary disease, pneumonia, or any combination thereof.

88. The method of claim 46, wherein said cancer health state comprises a cancer of stage I, II, or III.

89. The method of claim 46, wherein said trained predictive model is trained with a feature set and a health state of one or more subjects.

90. The method of claim 46, wherein said trained predictive model comprises a machine learning model, one or more machine learning models, an ensemble of machine learning models, or any combination thereof.

91. The method of claim 46, wherein said trained predictive model comprises a regularized machine learning model.

92. The method of claim 90, wherein said machine learning model comprises a machine learning classifier.

93. The method of claim 90, wherein said machine learning model comprises a gradient boosting machine, neural network, support vector machine, k-means, classification trees, random forest, regression, or any combination thereof machine learning models.

94. A system for diagnosing a cancerous or non-cancerous health state of one or more subjects, the system comprises:
(a) one or more processors; and
(b) a non-transitory computer readable storage medium including software configured to cause said one or more processors to:
(i) receive one or more subjects' one or more nucleic acid molecule sequencing reads of said one or more subjects' biological samples, wherein said one or more nucleic acid molecule sequencing reads comprise a sequence of an amplified one or more genomic features of one or more non-mammalian nucleic acid molecules; and
(ii) output a diagnosis of a cancerous or non-cancerous health state of said one or more subjects at least as a result of providing said one or more non-mammalian nucleic acid sequencing reads' one or more genomic features as an input to a trained predictive model.

95. The system of claim 94, wherein said non-mammalian nucleic acid molecules comprise microbial nucleic acids.

96. The system of claim 94, wherein said biological samples comprise a tissue, liquid, or any combination thereof biopsy samples.

97. The system of claim 94, wherein said one or more subjects comprise human, non-human mammal, or any combination thereof subjects.

98. The system of claim 94, wherein said one or more nucleic acid molecule sequencing reads comprise sequencing reads of DNA, RNA, cell-free DNA, cell-free RNA, exosomal DNA, exosomal RNA, cell-free microbial DNA, cell-free microbial RNA, or any combination thereof.

99. The system of claim 94, wherein said one or more genomic features comprise microbial phylogenetic marker genes or marker gene fragments thereof.

100. The system of claim 99, wherein said microbial phylogenetic marker genes comprise bacterial marker genes or marker gene fragments thereof.

101. The system of claim 99, wherein said microbial phylogenetic marker genes comprise fungal marker genes or marker gene fragments thereof.

102. The system of claim 100, wherein said bacterial marker genes comprise ribosomal RNA genes.

103. The system of claim 102, wherein said ribosomal RNA genes comprise 5S, 16S, 23S, or any combination thereof ribosomal RNA genes.

104. The system of claim 100, wherein said bacterial marker genes comprise: ribosomal RNA gene 5S, ribosomal RNA gene 16S, ribosomal RNA gene 23S, bacterial housekeeping genes dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, tsf, or any combination thereof.

105. The system of claim 101, wherein said fungal marker genes comprise: ribosomal RNA gene 18S, ribosomal RNA gene 5.8S, ribosomal RNA gene 28S, the internal transcribed spacer regions 1 and 2, or any combination thereof.

106. The system of claim 94, wherein said amplified one or more genomic features of said one or more non-mammalian nucleic acid molecules are amplified by polymerase chain reaction or derivatives thereof.

107. The system of claim 106, wherein said derivatives thereof comprise inverse PCR, anchored PCR, primer-directed rolling circle amplification, or any combination thereof.

108. The system of claim 106, wherein said polymerase chain reaction comprises blocking primers, marker gene primers, or any combination thereof, configured to prevent amplification of one or more genomic features.

109. The system of claim 108, wherein said one or more genomic features comprise mitochondrial DNA genomic features.

110. The system of claim 94, wherein said one or more nucleic acid molecule sequencing reads comprise sequencing reads of one or more enriched nucleic acid molecules.

111. The system of claim 110, wherein said one or more nucleic acid molecules of said one or more nucleic acid molecule sequencing reads comprise mammalian, non-mammalian, or any combination thereof nucleic acid molecule sequencing reads.

112. The system of claim 110, wherein said one or more enriched nucleic acid molecules are generated by:
(a) combining one or more nucleic acid molecules of said one or more nucleic acid molecule sequencing reads with hybridization probes, wherein said hybridization probes comprise a nucleic acid sequence complementarity to non-mammalian genomic features;
(b) incubating said hybridization probes and said one or more nucleic acid molecules under conditions that promote nucleic acid base pairing between target nucleic acid features of said one or more nucleic acid molecules and said hybridization probes;
(c) separating unbound hybridization probes and hybridized probes bound to one or more non-mammalian nucleic acid molecules; and
(d) washing said hybridized probes bound to said one or more non-mammalian nucleic acid molecules, thereby generating one or more enriched non-mammalian nucleic acid molecules.

113. The system of claim 112, wherein washing is configured to remove non-specifically associated nucleic acids and other reaction components.

114. The system of claim 110, wherein said one or more enriched nucleic acid molecules are generated by non-mammalian DNA enrichment.

115. The system of claim 114, wherein said non-mammalian DNA enrichment comprises:
(a) combining said one or more nucleic acid molecules with one or more recombinant CXXC-domain proteins to form a protein-DNA binding reaction;
(b) incubating said protein-DNA binding reaction under conditions that promote an interaction between said recombinant CXXC-domain proteins and non-methylated CpG motifs of said one or more nucleic acid molecules;
(c) separating unbound recombinant CXXC-domain proteins and recombinant CXXC-domain proteins bound to said non-methylated CpG nucleic acid molecule fragments from the remainder of said protein-DNA binding reaction;
(d) washing said recombinant CXXC-domain proteins bound to said non-methylated CpG nucleic acid fragments, thereby generating one or more enriched nucleic acid molecules for amplification.

116. The system of claim 115, wherein washing is configured to remove non-specifically associated nucleic acid molecules and said remainder of said protein-DNA binding reaction components.

117. The system of claim 94, wherein said software configures said one or more processors to filter said one or more nucleic acid molecule sequencing reads.

118. The system of claim 117, wherein said filtering comprises filtering said one or more nucleic acid molecule sequencing reads to produce one or more mitochondrial DNA-depleted non-mammalian sequencing reads.

119. The system of claim 118, wherein filtering comprises mapping said one or more mitochondrial DNA-depleted non-mammalian sequencing reads against one or more microbial reference databases to determine microbial taxonomic identity of said one or more mitochondrial DNA-depleted non-mammalian sequencing reads.

120. The system of claim 119, wherein said software configures said one or more processors to decontaminate said one or more mitochondrial DNA-depleted non-mammalian sequencing reads.

121. The system of claim 120, wherein decontaminating comprises in-silico decontamination.

122. The system of claim 120, wherein said decontaminating is configured to remove non-endogenous microbial sequencing reads, thereby generating decontaminated microbial taxonomic assignments and associated quantity of sequencing reads.

123. The system of claim 119, wherein mapping is performed with QIIME2 or other supported versions thereof.

124. The system of claim 119, wherein said one or more microbial reference databases comprise the bacterial 16S rRNA database Greengenes; the bacterial, fungal and archaeal rRNA database SILVA; the eukaryotic nuclear ribosomal ITS region database UNITE; a custom database derived from publicly available and complete microbial genome sequences; or any combination thereof.

125. The system of claim 111, wherein said amplified one or more genomic features comprise an abundances of one or more non-mammalian sequencing reads' microbial functional genes, biochemical pathways, or any combination thereof abundances.

126. The system of claim 122, wherein said software configures said one or more processors to predict metagenomic functional content of said decontaminated microbial taxonomic assignments, thereby producing one or more functional abundances.

127. The system of claim 126, wherein predicting said metagenomic functional content is performed by PICRUSt2.

128. The system of claim 94, wherein said biological samples comprise a tissue, liquid, or any combination thereof biopsy sample.

129. The system of claim 128, wherein said liquid biopsy sample comprises: plasma, serum, whole blood, urine, cerebral spinal fluid, saliva, sweat, tears, exhaled breath condensate, or any combination thereof.

130. The system of claim 94, wherein said cancerous health state comprises lung, breast, ovarian, gastro-intestinal, head and neck, liver, pancreas, prostate, skin, or any combination thereof cancers.

131. The system of claim 130, wherein said lung cancer comprises non-small cell lung cancer.

132. The system of claim 94, where said non-cancerous heath state comprises healthy, disease, or any combination thereof non-cancer state.

133. The system of claim 132, wherein said disease state comprises lung disease, wherein said lung disease comprises: carcinoid, hamartoma, granuloma, interstitial fibrosis, emphysema, bronchitis, chronic obstructive pulmonary disease, pneumonia, sarcoidosis, or any combination thereof.

134. The system of claim 94, wherein said cancerous health state comprises a cancer of stage I, II, or III.

135. The system of claim 94, wherein said trained predictive model comprises a machine learning model, one or more machine learning models, an ensemble of machine learning models, or any combination thereof.

136. The system of claim 94, wherein said trained predictive model comprises a regularized machine learning model.

137. The system of claim 135, wherein said machine learning model comprises a machine learning classifier.

138. The system of claim 135, wherein said machine learning model comprises a gradient boosting machine, neural network, support vector machine, k-means, classification trees, random forest, regression, or any combination thereof machine learning models.

139. The system of claim 94, wherein said cancerous health state or said non-cancerous health state comprise a category, tissue-specific location of cancer or disease, or any combination thereof.

140. The system of claim 94, wherein said cancerous health state comprises one or more types of cancer, one or more subtypes of cancer, stage of cancer, cancer prognosis, or any combination thereof.

141. The system of claim 94, wherein said trained predictive model is used to predict cancer therapy response of said one or more subjects.

142. The system of claim 94, wherein said trained predictive model is utilized to select an optimal therapy for a disease or cancer of said one or more subjects.

143. The system of claim 94, wherein said trained predictive model is utilized to longitudinally model a course of one or more cancers or one or more diseases of said one or more subjects' response to a therapy and to then adjust a treatment regimen.

144. The system of claim 94, wherein said cancerous health state comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof cancers.

145. The system of claim 94, wherein said trained predictive model is configured to remove contaminate non-mammalian features from said one or more nucleic acid molecule sequencing reads while selectively retaining sequences of non-contaminate non-mammalian features of said one or more non-mammalian nucleic acid molecules.

146. A method of generating a feature set for differentiating a cancer type of one or more subjects, the method comprising:
(a) providing one or more subjects' one or more nucleic acid molecules and corresponding health states;
(b) amplifying one or more genomic features of one or more non-mammalian nucleic acid molecules of said one or more nucleic acid molecules, thereby generating an amplified one or more genomic features;
(c) sequencing said amplified one or more genomic features to generate one or more non-mammalian sequencing reads; and
(d) generating a feature set configured to differentiate a cancer type by combining an abundances of said one or more genomic feature of said one or more non-mammalian sequencing reads and said health state of said one or more subjects.

147. The method of claim 146, wherein said one or more genomic features comprise microbial phylogenetic marker genes or marker gene fragments thereof.

148. The method of claim 147, wherein said microbial phylogenetic marker genes comprise bacterial marker genes or marker gene fragments thereof.

149. The method of claim 147, wherein said microbial phylogenetic marker genes comprise fungal marker genes or marker gene fragments thereof.

150. The method of claim 148, wherein said bacterial marker genes comprise: ribosomal RNA gene 5S, ribosomal RNA gene 16S, ribosomal RNA gene 23S, bacterial housekeeping genes dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, tsf, or any combination thereof.

151. The method of claim 149, wherein said fungal marker genes may comprise one or more of: ribosomal RNA gene 18S, ribosomal RNA gene 5.8S, ribosomal RNA gene 28S, and the internal transcribed spacer regions 1 and 2.

152. The method of claim 147, wherein microbial phylogenetic marker genes comprise bacterial, fungal, or any combination thereof marker genes.

153. The method of claim 146, wherein amplifying comprises performing a polymerase chain reaction or derivatives thereof.

154. The method of claim 153, wherein said derivatives thereof comprise inverse PCR, anchored PCR, primer-directed rolling circle amplification, or any combination thereof.

155. The method of claim 153, wherein said polymerase chain reaction comprises blocking primers, marker gene primers, or any combination thereof, configured to prevent amplification of one or more genomic features.

156. The method of claim 155, wherein said one or more genomic features comprise mitochondrial DNA genomic features.

157. The method of claim 146, comprising enriching said one or more nucleic acid molecules.

158. The method of claim 157, wherein said one or more nucleic acid molecules comprise mammalian, non-mammalian, or any combination thereof nucleic acid molecules.

159. The method of claim 158, wherein enriching comprises:
(a) combining said one or more mammalian nucleic acid molecules and said one or more non-mammalian nucleic acid molecules with hybridization probes, wherein said hybridization probes comprise a nucleic acid sequence complementarity to non-mammalian genomic features;
(b) incubating said hybridization probes and said one or more mammalian nucleic acid molecules and said one or more non-mammalian nucleic acid molecules under conditions that promote nucleic acid base pairing between target nucleic acid features of said one or more non-mammalian nucleic acid molecules and said hybridization probes;
(c) separating unbound hybridization probes and hybridized probes bound to said one or more non-mammalian nucleic acid molecules; and
(d) washing said hybridized probes bound to said one or more non-mammalian nucleic acid molecules, thereby generating one or more enriched non-mammalian nucleic acid molecules.

160. The method of claim 159, wherein washing is configured to remove non-specifically associated nucleic acid molecules and other reaction components.

161. The method of claim 158, wherein enrichment of said one or more nucleic acid molecules comprises non-mammalian DNA enrichment.

162. The method of claim 161, wherein said non-mammalian DNA enrichment comprises:
(a) combining said one or more mammalian nucleic acid molecules and said one or more non-mammalian nucleic acid molecules with one or more recombinant CXXC-domain proteins to form a protein-DNA binding reaction;
(b) incubating said protein-DNA binding reaction under conditions that promote an interaction between said recombinant CXXC-domain proteins and non-methylated CpG motifs of said one or more mammalian nucleic acid molecules or said one or more non-mammalian nucleic acid molecules;
(c) separating unbound recombinant CXXC-domain proteins and recombinant CXXC-domain proteins bound to said non-methylated CpG nucleic acid molecule fragments from a remainder of said protein-DNA binding reaction;
(d) washing said recombinant CXXC-domain proteins bound to said non-methylated CpG nucleic acid molecule fragments, thereby generating one or more enriched nucleic acid molecules for amplification.

163. The method of claim 162, wherein washing is configured to remove non-specifically associated nucleic acid molecules and said remainder of protein-DNA binding reaction components.

164. The method of claim 146, wherein said one or more nucleic acids are derived from one or more biological samples of said one or more subjects.

165. The method of claim 164, wherein said one or more biological samples comprise a tissue, liquid, or any combination thereof biopsy sample.

166. The method of claim 146, wherein said one or more subjects comprise human, non-human mammal, or any combination thereof subjects.

167. The method of claim 158, wherein said one or more mammalian nucleic acid molecules comprise DNA, RNA, cell-free DNA, cell-free RNA, exosomal DNA, exosomal RNA, or any combination thereof nucleic acids.

168. The method of claim 158, wherein said one or more non-mammalian nucleic acid molecules comprise microbial DNA, microbial RNA, microbial cell free DNA, microbial cell free RNA, or any combination thereof.

169. The method of claim 146, comprising filtering said one or more non-mammalian sequencing reads.

170. The method of claim 169, wherein filtering comprises filtering said one or more non-mammalian sequencing reads to produce one or more mitochondrial DNA-depleted non-mammalian sequencing reads.

171. The method of claim 170, wherein filtering comprises mapping said one or more mitochondrial DNA-depleted non-mammalian sequencing reads against one or more microbial reference databases to determine microbial taxonomic identity of said one or more mitochondrial DNA-depleted non-mammalian sequencing reads.

172. The method of claim 171, comprising decontaminating said one or more mitochondrial DNA-depleted non-mammalian sequencing reads.

173. The method of claim 172, wherein decontaminating comprises in-silico decontamination.

174. The method of claim 172, wherein decontaminating is configured to remove non-endogenous microbial sequencing reads, thereby generating decontaminated microbial taxonomic assignments and associated quantity of sequencing reads.

175. The method of claim 171, wherein mapping is performed with QIIME2 or other supported versions thereof.

176. The method of claim 171, wherein said one or more microbial reference databases comprise the bacterial 16S rRNA database Greengenes; the bacterial, fungal and archaeal rRNA database SILVA; the eukaryotic nuclear ribosomal ITS region database UNITE; a custom database derived from publicly available and complete microbial genome sequences, or any combination thereof.

177. The method of claim 146, wherein said one or more genomic feature abundances of said one or more non-mammalian sequencing reads comprise microbial functional gene, biochemical pathway, or any combination thereof abundances.

178. The method of claim 174, comprising predicting metagenomic functional content of said decontaminated microbial taxonomic assignments, thereby producing one or more functional abundances.

179. The method of claim 178, wherein predicting said metagenomic functional content is performed by PICRUSt2.

180. The method of claim 165, wherein said liquid biopsy sample comprises: plasma, serum, whole blood, urine, cerebral spinal fluid, saliva, sweat, tears, exhaled breath condensate, or any combination thereof.

181. The method of claim 146, wherein said cancer type comprises lung, breast, ovarian, gastro-intestinal, head and neck, liver, pancreas, prostate, skin, or any combination thereof cancers.

182. The method of claim 181, wherein said lung cancer comprises non-small cell lung cancer.

183. The method of claim 146, wherein said cancer type comprises a cancer of stage I, II, or III.

184. The method of claim 146, comprising generating a trained predictive model, wherein said trained predictive model is trained with said feature set and said health state of said one or more subjects.

185. The method of claim 184, wherein said trained predictive model comprises a machine learning model, one or more machine learning models, an ensemble of machine learning models, or any combination thereof.

186. The method of claim 184, wherein said trained predictive model comprises a regularized machine learning model.

187. The method of claim 185, wherein said machine learning model comprises a machine learning classifier.

188. The method of claim 185, wherein said machine learning model comprises a gradient boosting machine, neural network, support vector machine, k-means, classification trees, random forest, regression, or any combination thereof machine learning models.
